(19) United States
(12) Patent Application Publication
(10) Pub. No.: US 2011/0239327 A1
(43) Pub. Date: Sep. 29, 2011

(54) Nematicidal Effects of Cysteine Proteinases and Methods of Use Thereof to Treat Nematode Infestation

(76) Inventors: Rosane Curtis, Hertfordshire (GB); David Buttle, Sheffield (GB); Jerzy Behnke, Nottingham (GB); Ian Duce, Nottingham (GB); Peter Shewry, London (GB); Smita Kurup, Hertfordshire (GB); Brian Kerry, Herts (GB); Maxine Kerry, Herts (GB)

(21) Appl. No.: 12/521,450

(22) PCT Filed: Jan. 16, 2008
(86) PCT No.: PCT/IB2008/000522

§ 371 Date: Jan. 10, 2011

Related U.S. Application Data
(60) Provisional application No. 60/885,169, filed on Jan. 16, 2007.

(51) Int. Cl. A01N 47/12; A01N 37/18; C12N 15/82; C12N 15/63; A01H 5/00; A01P 5/00

(52) U.S. Cl. 800/279; 424/94.65; 435/320.1; 800/298

(57) ABSTRACT

This invention provides methods and compositions for control of nematicidal damage to economically important plants by contacting the nematodes with cysteine proteinases. The cysteine proteinases may be provided as crude plant extracts or as refined enzyme extracts. In addition, the cysteine proteinases may be provided by the plants expressing these enzymes. Furthermore, the cysteine proteinase treatment according to this invention may be utilized to potentiate the anti-nematode effects of a non-enzymatic nematicide.

This application claims priority to U.S. Provisional Application 60/885,169 filed Jan. 16, 2007, which is incorporated by reference herein as though set forth in full.

### FIELD OF THE INVENTION

This invention relates to the fields of plant biology and parasitology. More specifically, the invention provides means for the control of nematicidal damage to economically important plants by contacting the nematodes with cysteine proteinases.

### BACKGROUND OF THE INVENTION

Several publications and patent documents are cited throughout the specification in order to describe the state of the art to which this invention pertains. Each of these citations is incorporated by reference herein as though set forth in full.

Sedentary plant parasitic nematodes of the genera Meloidogyne and Globodera cause US$70 billion in crop losses worldwide each year, despite the use of control measures (Chitwood, 2003). Although there are several ways to manage these nematode pests in developed agricultural systems, protection relies on the use of crop rotation, resistant cultivars and the use of nematicides. Chemical control is expensive and is economic only on high value crops, and public health and environmental safety concerns over the use of these pesticides has led to the withdrawal of several products from the market (Chitwood, 2003; Meyer, 2003). The loss of these chemicals has therefore increased the need for novel methods of nematode management.

Nematode infection often presents without clear symptoms, thus, the economic effect of infection tends to be underestimated by growers. Notably, annual losses to world agriculture are estimated to be US$100 billion (Sasser & Freckman, 1987). Because nematode infestation frequently remains undetected due to this lack of symptoms, infestation is often severe when discovered (Atkinson, 1996). For example, in EU countries the combined annual yield loss to sugar beet cyst nematode is $95 million (Müller, 1999). Potato cyst nematodes occur in at least 64% of potato fields in England and Wales (Minnis et al., 2002) and are estimated to cause annual yield losses of approximately $80 million (Haydock and Evans, 1998). In 1998, the top ten soybean-producing countries, accounting for 97% of the world crop, suffered losses to soybean cyst nematode estimated at $1960 million (Wrather et al., 2001).

Control of cyst nematodes presents a particular problem as many or all of the eggs remain dormant within the protective cyst wall in soil for many years. Current control relies on a number of strategies, usually deployed in an integrated approach. Nematicides are widely used but some effective compounds have already been withdrawn due to their mammalian toxicity and environmental concerns. Of those chemicals remaining in use, the nematostat Aldicarb™ (Temik) will be withdrawn from use by the EU in 2007 but others such as Oxamyl™ (Vydate) and Fosthiazate™ (Nemathorin) will remain available. Nematicide use imposes substantial costs and is often not economic for some crops such as soybean. It adds over 50% to the variable costs associated with potato production in the UK.

Natural products with nematicidal properties have been identified by testing the effect of plant extracts (from leaves, stems, fruits and seeds), oil extracts, plant exudates and plant volatiles on nematodes that infect plants (Akhtar and Mahmood, 1994). Chopped shoots and plant extracts from plants, such as Carica papaya, Ficus carica, Ananas comosus, Plumeria rubra and Asclepias sinaica, were shown to be nematicidal to migratory endoparasitic nematodes and to reduce infection of plants (Mansoor et al. 1992; Sundaraju et al. 2003; Ahmad et al. 2004). However, the mode of action of most of these nematicidal extracts is unknown and the rates of application of most plant materials are too high to be useful in practice. Miller and Sands (1977) showed that the migratory ectoparasite Tylenchohynchus dubius was more susceptible to papain and bromelain than the migratory endoparasite Pratylenchus penetrans, suggesting that there may be significant differences between these two nematodes.

For centuries, the use of natural plant extracts for the treatment of gastrointestinal (GI) nematode infections of both humans and livestock has been part of traditional medicine worldwide, especially in developing countries (Giday et al. 2003). Of these plant extracts, the most studied appear to be those from the latex and fruit of plants such as papaya (C. papaya), fig (Ficus spp.) and pineapple (A. comosus). Extracts from papaya have been used against ascarids, tapeworms, whipworms and hookworms (Berger and Asenjo, 1940). Although the active constituent was not determined until much later, Robbins (1930) indicated that the mechanism of action of these extracts was to digest the cuticle. The latex of fig (Sgarbieri et al. 1964) and papaya (Dubois et al. 1988), and the fruit of pineapple (Rowan, Buttle and Barrett, 1990) and kiwi (Actinidia chinensis; McDowall, 1970) are known to contain proteolytic enzymes of the papain family of cysteine proteinases (Sub-family C1A of Family C01 in the Merops database on the world wide web at merops.sanger.ac.uk), such as papain from C. papaya, ficin from Ficus spp. and bromelain from A. comosus. Our previous studies, examining the in vitro and in vivo effects of the cysteine proteinases from papaya, fig, pineapple and kiwi fruit against three rodent GI nematodes, Heligmosomoides polygyrus in the small intestine (Stepek et al. 2005), Trichuris muris in the caecum (Stepek et al. 2006) and Protospirura muricola in the stomach (Stepek et al., 2007b) found that the active constituents were the cysteine proteinases, and that these enzymes from papaya, pineapple and fig, but not from kiwi fruit, had a rapid detrimental effect on the cuticle, which was completely digested, leading to the death of all three nematode species.

A number of studies using plant extracts are noted here:

Miller and Sands (1977) showed the ectoparasite Tylenchohynchus dubius was more susceptible to papain and bromelain than the migratory endoparasite Pratylenchus penetrans thereby providing an indication of major differences in the cuticle of these two nematodes.

Several studies have been conducted to identify natural products with nematicidal effects, by testing the effect of several plant extracts (e.g., from leaves, stems, certain fruits, seeds; oil extracts, plant exudates, plant volatiles, etc.) on nematode infection of plants. Chopped plant leaves and stems of several plants have also been incorporated into infested soil and the rate of nematode infection observed.

The mode of action of most plants containing or releasing nematicidal compounds that reduce nematode numbers has not yet been established.

A wide range of plants and plant extracts have also been used traditionally for the treatment of gastrointestinal nematodes. Researchers from Nottingham and Sheffield Universities analyzed the antihelmintic activity of a range of plant cysteine proteinases. Plant cysteine proteinases from fruits or latex of plants such as papaya, pineapple and fig have high proteolytic activities that are known to cause a reduction in motility and damage to the worms. The damage involves digestion and removal of the cuticle of gastrointestinal nematodes (Gillian et al., 2004; 2005).

The sedentary plant parasitic nematodes Meloidogyne sp. and Globodera sp. are amongst the world's most damaging agricultural pests attacking nearly all crops grown and potato crops, respectively. These nematodes develop a nutritional relationship with the host cells through the development of specific feeding sites. Current approaches to combat crop losses include nematicide application, cultural techniques and resistant cultivars used in an integrated manner. Alternative control is required urgently, because of growing health and environmental concerns over the use of toxic nematicides.

Migratory endoparasitic nematodes also cause significant damage to certain crops. For example, Radopholus similis is a worldwide pest in banana production. Turfgrasses are often infested with sting (Belaonolaimus sp.), needle, (Longidorus breviannulatus), lance (Hopololaimus galeatus) dagger, (Xiphinema americanum), stunt (Tylenchorhynchus ssp.) and spiral (Helicotylenchus pseudorobustus) nematodes which cause significant damage. All of these infestations are frequently treated with nematicide application. However, as above, alternatives to the nematicides currently available which are less toxic to humans and the environment are urgently needed.

### SUMMARY OF THE INVENTION

In accordance with the present invention, we have found that cysteine proteinases are active against parasitic nematodes. Thus, in one aspect, the invention provides a method for controlling nematode infestation of plants which comprises contacting nematodes with at least one plant cysteine proteinase or functional fragments thereof, plus sufficient cysteine or other suitable reducing agent to ensure activity of the cysteine proteinase. The method described above has utility for treatment of sedentary, migratory and animal parasitic nematodes. In one embodiment, the method is employed to control infestation of plants by sedentary plant parasitic nematodes. In another embodiment, control of lance or sting nematode infestation is achieved in turf grass using the compositions and methods described herein.

Cysteine proteinases isolated from kiwi and/or barley are particularly preferred for use in the method described above. The use of EP-B2 from barley is also preferred. In yet another aspect, the method may further entail treatment of nematode infestation with an effective amount of a non-enzymatic nematicide.

In another embodiment of the invention, control of nematode infestation is achieved via introduction, and expression of a nucleic acid encoding said at least one cysteine proteinase in a plant wherein the plant so produced expresses the proteinase in sufficient quantity to confer resistance to parasitism by nematodes when compared to a non-transgenic plant. In a preferred embodiment, the nucleic acid is isolated from kiwi or barley. Thus, the invention also provides a vector, plasmid, phage or construct comprising the cysteine proteinase encoding nucleic acid wherein expression of said proteinase in said plant is effective to inhibit growth of plant parasitic nematodes. The cysteine proteinase encoding nucleic acids may be operably linked to a tissue specific promoter to drive expression in targeted regions of the plant. In a preferred embodiment, the promoter drives expression of the cysteine proteinases described above in the roots of the plant. Transgenic plants so produced are also encompassed by the present invention.

In yet another aspect, a nematotoxic composition comprising at least one plant cysteine proteinase or active fragments thereof, a carrier and cysteine is disclosed. The composition may optionally comprise growing media or soil. Pretreatment, concurrent treatment or periodic treatment of soil with the compositions described herein also comprises an aspect of the invention.

Finally, a method for controlling nematode infestation is provided wherein said plants are contacted with said at least one cysteine protease at sub lethal levels, wherein said levels are effective to inhibit mobility or infective capacity of said nematode.

### BRIEF DESCRIPTION OF THE DRAWINGS

FIG. 1: The number of active Meloidogyne spp. J2s after exposure to increasing active enzyme concentrations of papain over time. The proportion of active worms decreased linearly over the range of papain concentrations tested, while no change in activity was evident for the worms incubated in distilled water, with and without cysteine. Error bars represent the standard error of the mean.

FIG. 2: Light micrographs of Meloidogyne spp. J2s incubated with and without papain for 3 h. Digestion of the cuticle was evident at the anterior end of the nematodes incubated with 6.25 μM (A) and 100 μM (Bii) (active enzyme) papain. The J2s incubated with 100 μM papain (Bi) also showed an abnormal granular appearance of the internal structures. The J2s incubated without enzyme (C) had a normal appearance. Scale bar=10 μm.

FIG. 3: Scanning electron micrographs of Meloidogyne spp. J2s exposed to papain or actinidin for 4 h. Digestion of the cuticle was evident for the J2s incubated with 10004 (active enzyme) papain (Ai) and 100 μM (active enzyme) actinidin (Bi), but not for those incubated without enzyme (C). Note the absence of a distinct cuticular layer in the J2s incubated with papain (Aii) and actinidin (Bii), compared to J2s incubated without enzyme (Cii). Scale bars=1 μm (Ai, Aii, Bi, Bii, Ci, Cii), 10 μm (A), 100 μm (B, C).

FIG. 4: The number of active Meloidogyne spp. J2s after exposure to 50 μM (active enzyme) papain, with and without the cysteine proteinase-specific inhibitor, E-64 (1 mM). A difference in the proportion of active worms was observed between the J2s incubated with papain and the J2s incubated with papain which had been pre-incubated with E-64. No change in the proportion of active worms was evident on the addition of E-64 for J2s incubated without enzyme (data not shown). Error bars represent the standard error of the mean.

FIG. 5: The number of active Meloidogyne spp. J2s after exposure to 25 μM (active enzyme) papain, 25 μM (active enzyme) papaya latex and 25 μM (active enzyme) stem bromelain. Reductions in the proportion of active worms were evident for the J2s incubated with papain, papaya latex and stem bromelain, but not for the J2s incubated without enzyme. Error bars represent the standard error of the mean.

FIG. 6: The number of active G. rostochiensis J2s after exposure to 25 μM (active enzyme) papain, 25 μM (active enzyme) papaya latex and 25 μM (active enzyme) stem bromelain. Reductions in the proportion of active worms were evident for the J2s incubated with papaya latex and papain, but not with stem bromelain or for those J2s incubated without enzyme. Error bars represent the standard error of the mean.

FIG. 7: Light micrographs of Meloidogyne spp. adult female worms incubated with and without papain for 4 h. Digestion of the cuticle was evident for the nematodes incubated with 25 μM (active enzyme) papain (A). Note the release of the internal structures (arrowheads) from these nematodes and the cuticle coming away from the worm (arrows). The worms incubated without enzyme (B) had a normal appearance. Scale bar=10 μm.

FIG. 8: A graph showing the effect of papaya latex on M. incognita.

FIG. 9: A graph showing the effect of bromelain on M. incognita.

FIG. 10: A pair of graphs showing the effect of papain (FIG. 10A) and bromelain (FIG. 10B) on P. penetrans viability.

FIG. 11: A graph showing the effects of papain on nematodes in different soil compositions.

FIG. 12: A graph showing the effect on nematodes of soil treated with papain.

FIG. 13: A graph showing the effect on nematodes of soil treatment with papain and papain plus vydate.

FIG. 14: A graph showing the effects on nematodes of two soil treatments with cysteine proteinase.

FIG. 15: A graph showing the effects on nematodes of two soil treatments with cysteine proteinase with and without non-enzymatic nematicides

FIG. 16: A series of micrographs showing EP-B2 mediated destruction of the nematode cuticle. FIG. 16a) shows M. incognita J2 in after incubation for 16 hs in assay buffer pH 4 containing no enzyme. FIGS. 16b) and 16c) show M. incognita J2 after incubation with EP-B2 in assay buffer pH 4 for 16 hs, the enzyme affected the cuticle region around the nematode head (a) and tail (b).

### DETAILED DESCRIPTION OF THE PREFERRED EMBODIMENTS OF THE INVENTION

This invention relates to compositions and methods which are effective for control of plant parasitic nematodes. Specifically, plant cysteine proteinases isolated from papaya latex, papain, stem bromelain, kiwi fruits have been shown to effectively reduce nematode infestation of host plants and thus can be used to control nematode infestation and growth. We have found that certain natural plant cysteine proteinases produce progressive damage to the cuticle of plant parasitic nematodes Meloidogyne sp and Globodera sp. The mechanism of action of these plant enzymes is not completely understood, however an attack on the protective cuticle of the nematode is evident which involves blistering and eventually, total digestion of the cuticle. The cuticular protein(s) sensitive to digestion by the plant cysteine proteinases are not known, but from this work, they represent potential targets for the developing of novel measures to control plant parasitic nematodes. Application of cysteine proteases should also be effective to treat other types of nematode infestations, including Radopholus similis, Belaonolaimus sp., Hopolaimus sp., Longidorus breviannulatus, galeatus, Xiphinema americanum, Tylenchorhynchus ssp. and spiral Helicotylenchus pseudorobustus nematodes which also cause significant crop and turfgrass damage.

The enzymes disclosed herein have similar but not identical activities. They were not effective against the free living nematode Caenorhabditis elegans. Also, distinct activity has been observed since kiwi fruit extracts showed no effect in nematode motility or cuticle damage of animal parasitic nematodes (Stepek et al, 2004). However, the same extracts digested the cuticle of plant parasitic nematodes. These results indicate that there are distinct differences in the composition of the cuticle of these nematodes.

This is the first time that the nematicidal effect of natural plant cysteine proteinases from fruits is demonstrated for sedentary plant parasitic nematodes and for specific applications of migratory plant parasitic nematodes. We envisage that at lower concentrations these enzymes will affect nematode motility and therefore affect nematode infection of plants. Previous research in Rothamsted has shown that by affecting nematode motility we can interfere with nematode infection of plants (Fioretti, et al., 2002; Sharon, et al., 2002; GB Patent Filing 46082).

We have also discovered that EP-B2, a cysteine protease isolated from barley, (Behune et al., 2006; Davy et al., 1998) is nematotoxic. Accordingly, methods of use of this proteinase to control nematode infestation form an aspect of the invention.

Thus, in accordance with the present invention, a novel class of nematicides for controlling plant parasitic nematodes and a novel class of plants resistant to nematode infection by virtue of controlled expression of the cysteine proteinases are provided.

When the compositions described herein are applied as a bionematicide in the soil, the nematodes are not able to reach their host plants. In transformed plants secreting cysteine proteinases the nematodes are not able to migrate inside the root tissue and die before formation of their feeding site.

In accordance with the present invention, we have shown that cysteine proteinases from papaya, pineapple, kiwi fruit and barley cause substantial damage to the cuticle of sedentary plant parasitic nematodes (Meloidogyne spp. and G. rostochiensis), which resulted in a significant reduction in the activity of their second-stage juveniles Stepek et al., 2007a. This effect was similar to that described previously for gastrointestinal nematodes of humans and livestock (Robbins, 1930; Berger and Asenjo, 1939; Stepek et al. 2005, 2006, 2007b, indicating that certain plant cysteine proteinases have a broad spectrum of action against plant parasitic nematodes. These natural plant products showed efficacy against Meloidogyne spp., and similar effects were observed using the same concentrations and duration of incubation with papaya latex and papain, but not with stem bromelain, on G. rostochiensis J2s. It appears that longer incubation with papain was needed to cause some damage to the cuticle of G. rostochiensis; this would suggest that there are differences in the cuticle of these nematodes and that root-knot nematodes may be more susceptible to plant cysteine proteinases than potato cyst nematodes. This is probably due to the much tougher cuticle of G. rostochiensis second-stage juvenile, thus greater enzyme concentrations and/or longer incubation periods may be required for potato cyst nematodes to succumb to the detrimental effects of stem bromelain.

While not wishing to be bound by theory, it appears that the mechanism of action of plant cysteine proteinases against animal and plant nematodes is digestion of the cuticle, releasing the internal structures, leading to death of the parasites; however, the cuticular proteins susceptible to this digestion still remain unknown. Nevertheless, despite the similarity in effects of plant cysteine proteinases against parasitic nematodes of animals and plants, there are distinct differences. For instance, there is a much more rapid effect against animal nematodes (Stepek et al. 2005, 2006, 2007b) and there is a lack of efficacy of actinidin against animal nematodes but not against plant nematodes (Stepek et al. 2007a). The crystal structures of papain and actinidin are closely similar and virtually superimposable in the active-centre; however, there is evidence for differences in the characteristics and behaviour of their catalytic sites (Kowlessur et al. 1989). This indicates that the cuticles of animal and plant nematodes differ and that the susceptible cuticular proteins are likely to be different, based on the differential effect of actinidin on plant and animal nematodes.

Although the cysteine proteinases had nematicidal activity against the J2s and adult worms, we found no in vitro effects on the eggs of Meloidogyne spp. The structure of the eggs remained unaffected, even after overnight incubation with papain. This suggests that the components of the cuticle that are sensitive to attack by the cysteine proteinases are only present or exposed to the action of these enzymes in the infective stages and adult worms (Stepek et al., 2007a). This was also observed in the case of the rodent gastrointestinal nematode, Heligmosomoides polygyrus, in that the eggs of this nematode were not affected detrimentally by plant cysteine proteinases, and the structure and development of the L1 stage remained unaffected (Stepek et al. unpublished).

In a preferred embodiment of this invention, plants are contacted with plant debris from kiwi, papaya, barley and the like as disclosed herein which have demonstrable effective nematocidal activity. Alternatively, plant extracts containing the active cysteine proteinase (see for example U.S. Pat. No. 5,106,621, herein incorporated by reference), are added to soil in which plants to be protected against nematode pests are to be grown. In practice, it is desirable to deliver concentrations of the proteinases that are nematicidal. However, non-lethal effects on the surface coat of nematodes should have profound effects on host recognition and successful invasion. Thus another aspect of the invention entails delivery of sub-lethal levels of the cysteine proteinases described herein.

In an alternate embodiment of this invention, transgenic plants are produced which comprise heterologous nucleic acids encoding the cysteine proteinases described herein. Such plants should exhibit resistance to nematode infection. Transgenic turf grasses are a particularly preferred embodiment.

### DEFINITIONS

The plant parasitic nematodes referred to herein can be classified as ectoparasites which feed from the root exterior and endoparasites which live and feed inside the host plant.

Ectoparasites can be migratory or sedentary, they exist in soil and feed from the exterior of root by inserting stylet into plant tissue. They remain vermiform throughout the lifecycle. Some nematodes are vectors of plant viruses.

Migratory ectoparasites have the most primitive mode of parasitism and they remain outside the root and use their long stylet to feed from root cells. Examples include without limitation, Belonolaimus, and Tylenchorhynchus.

Sedentary ectoparasites will feed from a single site or plant cell for a prolonged period of time while remaining outside the root. Examples include, without limitation, Criconemella xenoplax and Hemicycliophora arenaria.

Plant parasitic genera of ectoparasites to be treated with the compositions of the invention include, for example, Belonolaimus, Paratylenchus, Criconemella, Helicotylenchus, Trichodorus, Hoplolaimus, Xiphinema, Tylenchorhynchus, Longidorus, Rotylenchus, Scutellonema, Hemicycliophora. Several of these species of nematodes cause significant damage to turf grasses.

Endoparasites exist in the soil for some period of time during their life cycles. They penetrate the root tissue to feed. Some feed as they become sedentary (Meloidogyne, Glododera) and others feed as they migrate inside the root tissue. (Pratylenchus).

Sedentary endoparasites feed by forming feeding sites (giant cells and syncytia) in the roots. These nematodes invade roots as vermiform second-stage juveniles (J2) and their development depends on the formation of specialised feeding cell that become the permanent source of nutrients for these parasites.

Migratory endoparasites periodically feed as they migrate intracellularly within the cortex, leaving tracks of destroyed cells behind. Examples include, Pratylenchus, Hirschmanniella and Radopholus.

The genera of endoparasites to be treated with the compositions of the invention include Hirchmaniella, Pratylenchus, Radopholus, Ditylenchus, Anguina, Aphelenchoides, Bursaphelenchus, Rhadinaphelenchus, Heterodera, Rotylenchus, Tylenchulus, Nacobbus, Globodera, and Meloidogyne.

Shoot Parasitic Nematodes feed on above ground plant tissue stems, bulbs, leaves, seeds and are mostly endoparasitic nematodes.

The most damaging plant parasitic nematodes include, Meloidogyne—Root-knot nematodes; Pratylenchus—lesion nematode; Heterodera—cyst nematode; Globodera—cyst nematode; Ditylenchus—stem and bulb nematode; Tylenchulus—citrus nematode; Xiphinema—dagger nematode; Radopholus—burrowing nematode; Rotylenchulus—reniform nematode; Helicotylenchus—spiral nematode; and Belonolaimus—sting nematodes.

“Plant” species of interest include, but are not limited to, corn (Zea mays), alfalfa (Medicago sativa), rice (Oryza sativa), rye (Secale cereale), sorghum (Sorghum bicolor, Sorghum vulgare), millet (e.g., pearl millet (Pennisetum glaucum)), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, grasses (particularly those found on golf courses) and conifers.

“Plant material” refers to leaves, stems, roots, flowers or flower parts, fruits, pollen, pollen tubes, ovules, embryo sacs, egg cells, zygotes, embryos, seeds, cuttings, cell or tissue cultures, or any other part or product of a plant.

“Nematotoxic growing media or soil” refers to growing media and/or soil that has been treated with the compositions described herein.

A “reducing agent” is a substance that chemically reduces other substances, especially by donating an electron or electrons. Suitable reducing agents for use in the invention include, without limitation, cysteine, Dithiothreitol (DTT), beta-mercaptoethanol (beta-me), and Tris(2-Carboxyethyl)-Phosphine Hydrochloride (TCEP HCl) are sulfhydryl protective reducing agents. Reducing agents are typically used to prevent the oxidation of free sulfhydryl residues (cysteines) in the protein.

As used herein, “transgenic plant” includes reference to a plant that comprises within its nuclear genome a heterologous polynucleotide. Generally, the heterologous polynucleotide encoding the desired cysteine protease is stably integrated within the nuclear genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic.

The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

The terms “percent similarity”, “percent identity” and “percent homology” when referring to a particular sequence are used as set forth in the University of Wisconsin GCG software program.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

A “replicon” is any genetic element, for example, a plasmid, cosmid, bacmid, plastid, phage or virus that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The phrase “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by calorimetric, fluorogenic, chemiluminescent, histochemical or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell or plant. A number of “selectable marker genes” are known in the art and several antibiotic resistance markers satisfy these criteria, including those resistant to kanamycin (nptII), hygromycin B (aph IV) and gentamycin (aac3 and aacC4). Useful dominant selectable marker genes include genes encoding antibiotic resistance genes (e.g., resistance to hygromycin, kanamycin, bleomycin, G418, streptomycin or spectinomycin); and herbicide resistance genes (e.g., phosphinothricin acetyltransferase). A useful strategy for selection of transformants for herbicide resistance is described, e.g., in Vasil, Cell Culture and Somatic Cell Genetics of Plants, Vols. I III, Laboratory Procedures and Their Applications Academic Press, New York, 1984. Particularly preferred selectable marker genes for use in the present invention would genes which confer resistance to compounds such as antibiotics like kanamycin, and herbicides like glyphosate (Della-Cioppa et al., Bio/Technology 5(6), 1987, U.S. Pat. Nos. 5,463,175, 5,633,435). Other selection devices can also be implemented and would still fall within the scope of the present invention.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

“Native” refers to a naturally occurring (“wild-type”) nucleic acid sequence.

“Heterologous” sequence refers to a sequence which originates from a foreign source or species or, if from the same source, is modified from its original form.

A “coding sequence” or “coding region” refers to a nucleic acid molecule having sequence information necessary to produce a gene product, when the sequence is expressed.

The 3′ non-translated region of the cysteine protease encoding gene constructs of the invention contain a transcriptional terminator, or an element having equivalent function, and, optionally, a polyadenylation signal, which functions in plants to cause the addition of polyadenylated nucleotides to the 3′ end of the RNA. Examples of suitable 3′ regions are (1) the 3′ transcribed, non-translated regions containing the polyadenylation signal of Agrobacterium tumor-inducing (Ti) plasmid genes, such as the nopaline synthase (NOS) gene, and (2) plant genes such as the soybean storage protein genes and the small subunit of the ribulose-1,5-bisphosphate carboxylase (ssRUBISCO) gene. An example of another 3′ region is that from the ssRUBISCO E9 gene from pea (European Patent Application 385,962, herein incorporated by reference in its entirety).

Such cysteine proteinases expressing plants may also comprise a selectable marker gene. Selectable marker genes are those that when expressed, confer a selectable phenotype, such as antibiotic resistance, on a transformed cell or plant. A number of “selectable marker genes” are known in the art and several antibiotic resistance markers satisfy these criteria, including those resistant to kanamycin (nptII), hygromycin B (aph IV) and gentamycin (aac3 and aacC4). Useful dominant selectable marker genes include genes encoding antibiotic resistance genes (e.g., resistance to hygromycin, kanamycin, bleomycin, G418, streptomycin or spectinomycin); and herbicide resistance genes (e.g., phosphinothricin acetyltransferase). A useful strategy for selection of transformants for herbicide resistance is described, e.g., in Vasil, Cell Culture and Somatic Cell Genetics of Plants, Vols. I III, Laboratory Procedures and Their Applications Academic Press, New York, 1984. Particularly preferred selectable marker genes for use in the present invention are genes which confer resistance to compounds such as antibiotics like kanamycin, and herbicides like glyphosate (Della-Cioppa et al., Bio/Technology 5(6), 1987, U.S. Pat. Nos. 5,463,175, 5,633,435). Other selection strategies can also be implemented and would still fall within the scope of the present invention.

### EXAMPLES

Having disclosed in general and specific terms the advantages of the present invention, including methods of making and using the invention and the best mode of conducting the invention, the following specific examples are provided to further extend the written description of the invention and to ensure that those skilled in the art are enabled to practice the invention. The invention, however, should not be considered to be limited by the specifics of these examples. Rather, for purposes of evaluating the scope of this invention, reference should be had to the appended claims and their equivalents.

### Materials and Methods

### Nematodes

Eggs of M. incognita race 1 from North Carolina State University, USA, and M. javanica from Portugal were collected from the roots of infected tomato plants grown in a glasshouse at 23° C. in a 16:8 h light-dark regime. Eggs were extracted using 1% hypochlorite (NaClO) solution (Hussey and Barker, 1973) or by picking egg masses from the infected roots. M. incognita and M. javanica were both used in the experiments and produced similar results. Juveniles (J2) were hatched from the eggs and egg masses in water. Globodera rostochiensis cysts were washed extensively in distilled water. Hatched J2s were obtained by incubating cysts in distilled water for 4 days followed by exposure to potato root diffusate (PRD).

### Enzymes

The enzymes used during this study were pure papain from the papaya plant (Carica papaya) (Merops identifier C01.001; Sigma catalogue number P3125), crude latex from papaya (C. papaya) (Sigma catalogue number P3250; lot number 124K1004), stem bromelain from the stem of the pineapple plant (Ananas comosus) (Merops identifier C01.005; Hong Mao Biochemicals Company, Thailand), and actinidin from acetone precipitation of kiwi fruit juice (Actinidia chinensis; Merops identifier C01.007). These enzyme preparations were standardised for the molar concentration of active cysteine proteinase by titration with the cysteine proteinase-specific inhibitor, L-trans-epoxysuccinyl-leucylamido(4-guanidino)butane (E-64) (Sigma catalogue number E3132) (Stepek et al. 2005). While purified enzymes were principally utilized in these examples, those skilled in the art will recognize that for field applications, crude enzymes may be used, debris from the relevant plants may be used. Indeed, other latex bearing plants containing cysteine proteinases could also be used to control plant parasitic nematodes. Plants other than those specifically mentioned herein which are latex bearing plants that have the same effect on nematodes could be more viable to use for field applications.

### Example 1

### In Vitro Assessment of the Activity of Plant Cysteine Proteinases Against Plant Parasitic Nematodes

Approximately 1000 J2s (second juvenile stage) of M. incognita or M. javanica were incubated per well of a 48-well plate with the following active enzyme concentrations (determined by E-64 titration) of papain dissolved in distilled water containing 16 mM L-cysteine (to activate the cysteine proteinase): 1.5 μM, 3 μM, 6.25 μM, 12.5 μM, 25 μM, 50 μM, 75 μM and 100 μM. Control wells contained J2s and distilled water with and without 16 mM L-cysteine, or J2s and 1 mM E-64, with and without 50 μM (active enzyme) papain, which had been preincubated for 25 mins at 40° C. Each enzyme concentration and the controls were set up in duplicate (n=2) or triplicate (n=3). The number of active J2s per well was counted prior to the addition of papain, and then the J2s were incubated in the dark at room temperature for 3 h. The number of mobile J2s per well was counted after 60, 120 and 180 mins. The nematode surfaces were examined for signs of damage using a light microscope (Zeiss Axioskop) at ×20, ×40 and ×100 magnification and photographs were taken with a Xillix Microimager. The same procedure was followed when setting up further experiments to compare the effects of 25 μM papain, 25 μM crude papaya latex (0.625 g/ml), 25 μM stem bromelain (0.18 g/ml) and 25 μM actinidin (active enzyme concentrations) with M. incognita, M. javanica or G. rostochiensis.

The effects on the J2 cuticle were more closely examined by scanning electron microscopy (SEM). Approximately 2000 J2s were used per enzyme or control treatment. These J2s were added to each of four 5 ml tubes and centrifuged at 2500 rpm 4° C. for 5 mins. The supernatant was removed and the J2s were resuspended with 500 μl of 100 μM papain, 100 μM actinidin (active enzyme concentrations) or distilled water with and without 16 mM L-cysteine (the enzyme dilutions were prepared with 16 mM L-cysteine dissolved in distilled water). The nematodes were incubated in the dark at ambient temperature for 4 h before centrifugation as above. The J2s were immediately fixed with 2.5% glutaraldehyde in 0.15M phosphate buffer, pH 7.2 for 1 h at room temperature and were then centrifuged as before prior to washing for 1 h in 0.15M phosphate buffer, pH 7.2 at 4° C. These J2s were then prepared for SEM as described previously by Stepek et al. (2005).

To assess whether these enzymes also affected the cuticles of other stages of the nematodes, five adult females of M. incognita were incubated in wells of a 48-well plate containing 25 μM (active enzyme) papain, prepared in 16 mM L-cysteine dissolved in phosphate buffered saline (PBS), pH 7.4, or PBS, pH 7.4 with and without 16 mM L-cysteine. These nematodes were incubated in the dark at ambient temperature for 4 h and were then examined using a light microscope at ×10 and ×20 magnification to observe any differences to the nematode body wall.

To determine if the enzymes affected the eggshell and/or the development and hatching of the juveniles which the eggs contain, approximately 300 Meloidogyne spp. eggs were incubated per well of a 48-well plate, in duplicate, in 50 μM (active enzyme) papain, prepared in 16 mM L-cysteine dissolved in distilled water, or distilled water with and without 16 mM L-cysteine. The number of eggs in each well was counted before the addition of papain and then the eggs were incubated in the dark at room temperature for 4 h. The number of eggs was counted before further incubation overnight. Finally, closer examination of the structure of the nematode eggs was performed using the light microscope at ×40 magnification.

### Statistics

The data from the in vitro motility experiments were analysed using a repeated measures analysis of variance (as implemented in the GenStat procedure AREPMEASUREMENTS) to compare the overall treatment means, the overall means at each sample time, and the treatment by time interaction. Numbers of nematodes moving at each of the repeated sampling times, 60, 120 and 180 mins, were expressed as a proportion of those moving initially at time 0 (i.e. this latter number was assumed to be the actual number of nematodes initially in the dish). The proportions were transformed to logits prior to analysis. A small adjustment (p=(r+0.5)/(n+1), where r is the number moving at a given time and n is the number moving at time 0) was made to all the data prior to transformation to allow for occurrences of 100%. Degrees of freedom were partitioned to explore particular treatment comparisons and trends, and were adjusted before testing the main effect of time and interaction terms to allow for unequal patterns of correlation due to the repeated nature of measurements on wells.

### Results

Effect of Plant Cysteine Proteinases on the Mobility of Meloidogyne spp. J2s

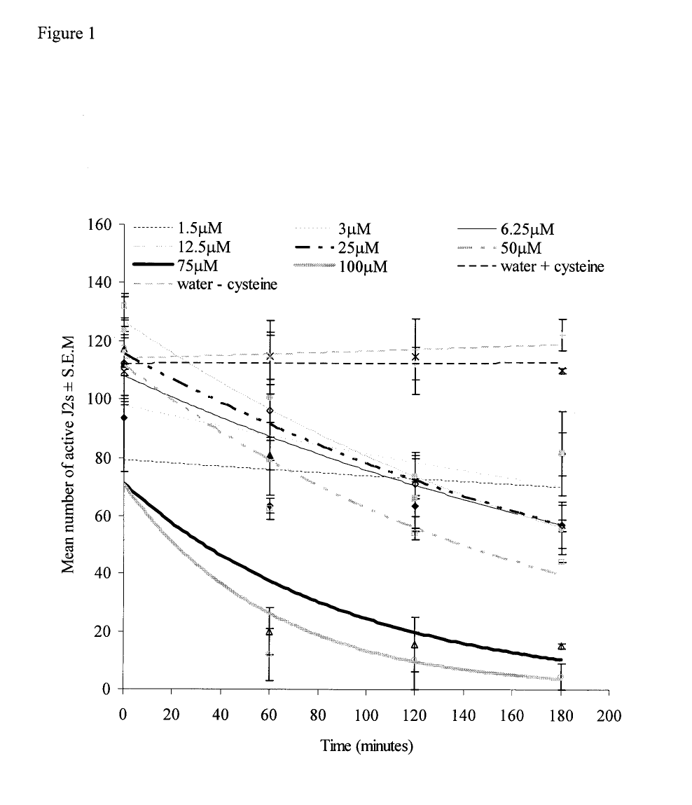

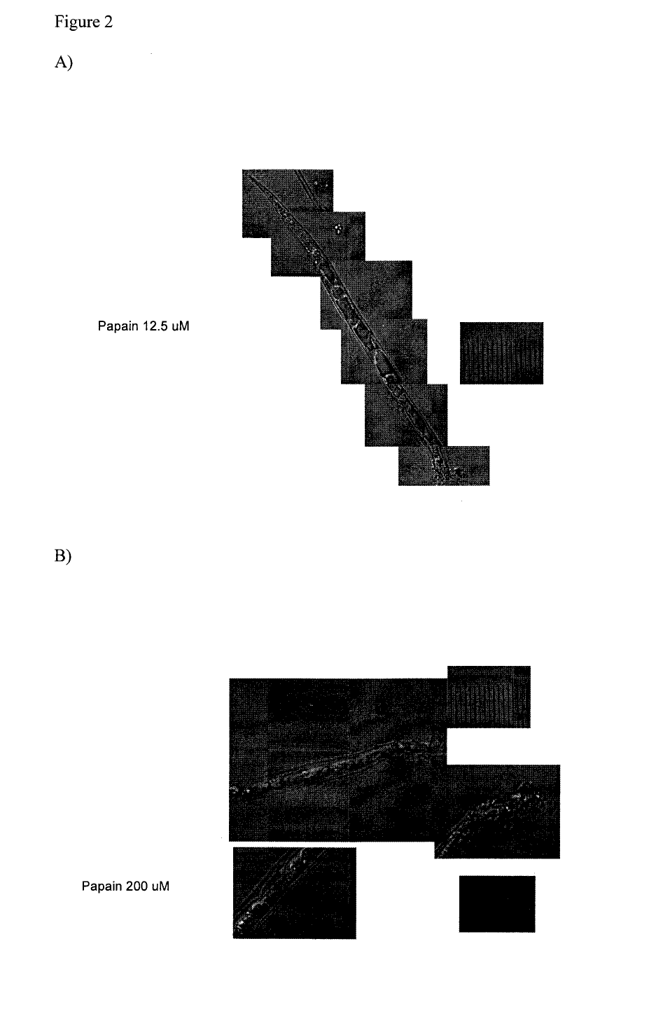

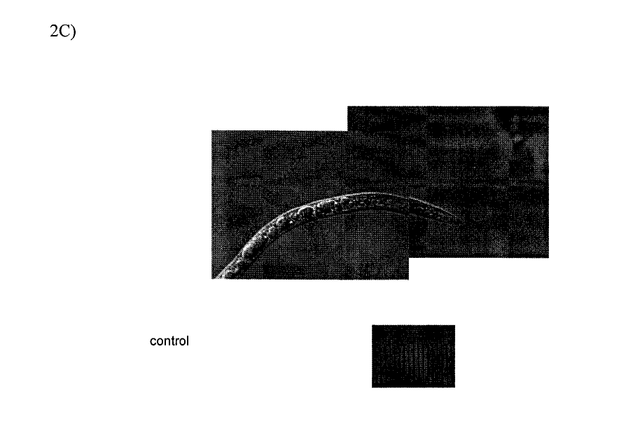

Nematode mobility was similar for the two controls (nematodes in distilled water, with and without cysteine; F_{1,10}=0.02, p=0.886), which was consistent over time (F_{1.5,14.7}=3.57, p=0.066). The control nematodes had a normal appearance. Addition of papain (data for all doses combined) decreased the mobility of Meloidogyne spp. J2s compared to the combined controls (F₁₁₀=65.1, p<0.001). As the concentration of papain increased, the proportion of mobile J2s of Meloidogyne spp. decreased linearly (F_{1,10}=14.0, p=0.004) (FIG. 1). Mobility was unaffected by time (F_{1.5,14.7}=1.5, p=0.255) and similar linear patterns with increasing papain concentration were observed at each time (F_{1.5,14.7}=0.5, p=0.558). Damage to the nematode surface also increased. Papain at active enzyme concentrations of 1.5 μM and 3 μM caused reductions in the activity of Meloidogyne J2s (e.g. 1.5 μM: t₁₆=2.99, p<0.001) relative to the combined controls, but there were no visible effects on the surface of the nematodes. Only active enzyme concentrations above 6.25 μM caused blistering of the cuticle, visible at ×40 and ×100 magnifications, at the anterior end of the nematode and an abnormal granular appearance of the internal structures (FIG. 2).

Plant Cysteine Proteinases Damage the Surface of Meloidogyne spp. J2s

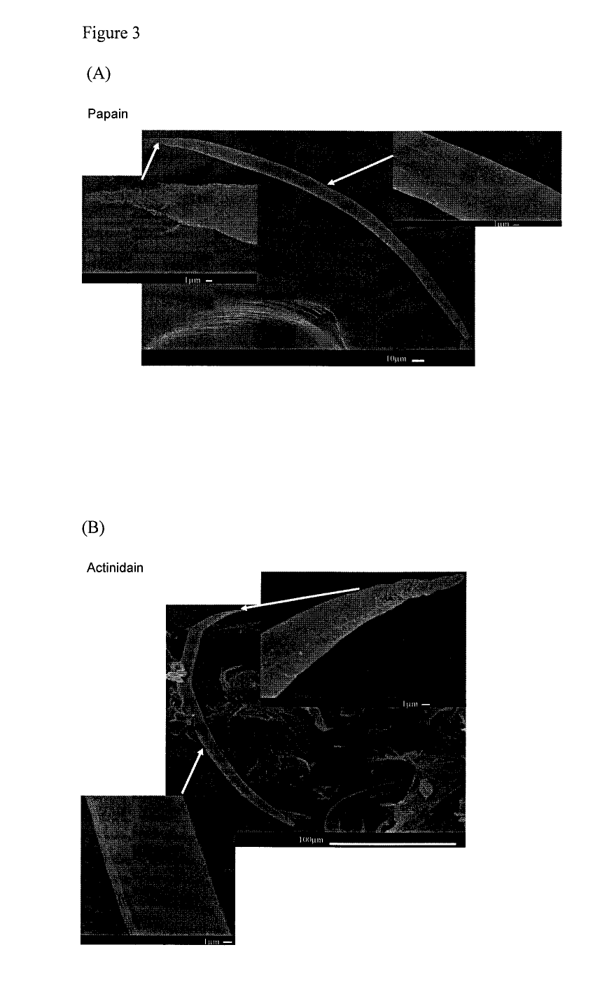

The surface damage observed with light microscopy was examined more closely using SEM. The surface cuticle of the J2s incubated with papain (FIG. 3A) and actinidin (FIG. 3B) had been digested, compared to those J2s not exposed to any enzyme (FIG. 3C), which retained a distinct cuticle layer (FIG. 3Cii). This layer had disappeared after 4 hours incubation with either papain (FIG. 3Aii) or actinidin (FIG. 3Bii).

Effect of Co-Incubation of Plant Cysteine Proteinases with the Cysteine Proteinase-Specific Inhibitor, E-64, on the Motility of Meloidogyne spp. J2s

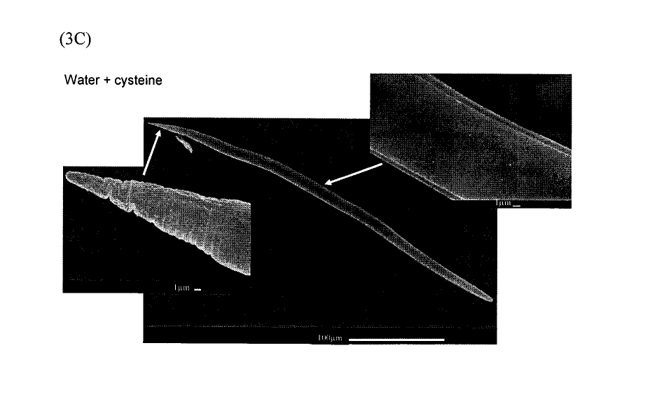

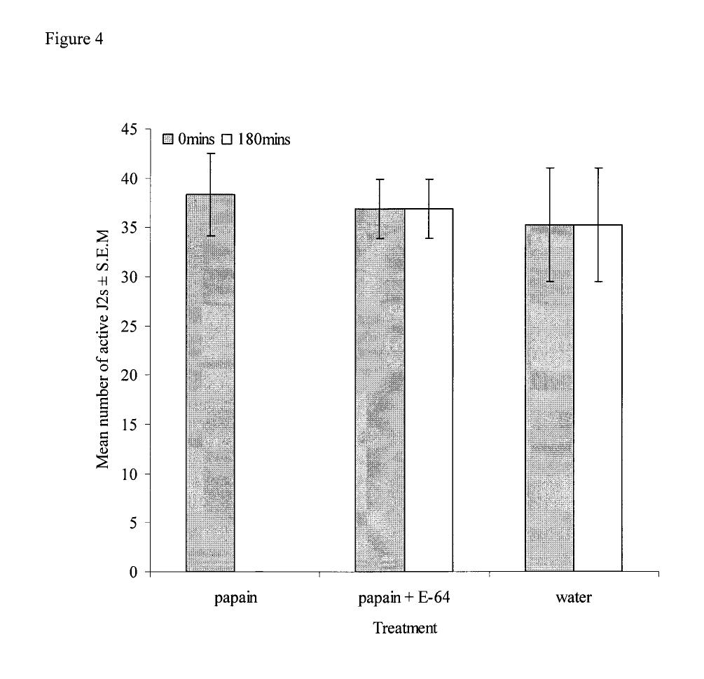

None of the 115 nematodes (n=3, mean=38.3) treated with papain alone were mobile after 180 mins, whereas all of the 111 nematodes (n=3, mean=37) treated with papain pre-incubated with the inhibitor, E-64, and all of the 106 nematodes (n=3, mean=35.3) treated with only water and cysteine were still mobile after this time (FIG. 4). This shows that there was an overall reduction in nematode mobility when treated with papain compared to no papain, but there was no reduction in the mobility of those nematodes exposed to papain which had been pre-incubated with the cysteine proteinase-specific inhibitor, E-64. Thus, the effects on the number of active worms and on the nematode surface were dependent upon the activity of the cysteine proteinases.

The nematicidal effect was not only caused by papain (FIG. 5) as a significant reduction (F_{1,4}=47.09, p=0.002) in the proportion of active nematodes relative to the control was also evident for the J2s incubated with papaya latex and stem bromelain (combined data). No differences in mobility were observed amongst the three enzymes (F_{2,4}=4.83, p=0.086).

Effect of Plant Cysteine Proteinases on the Mobility of G. rostochiensis J2s

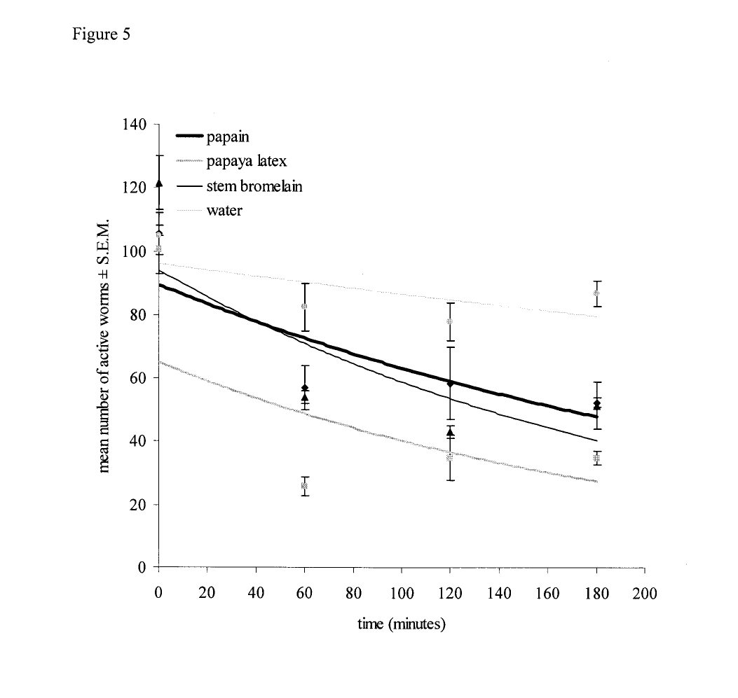

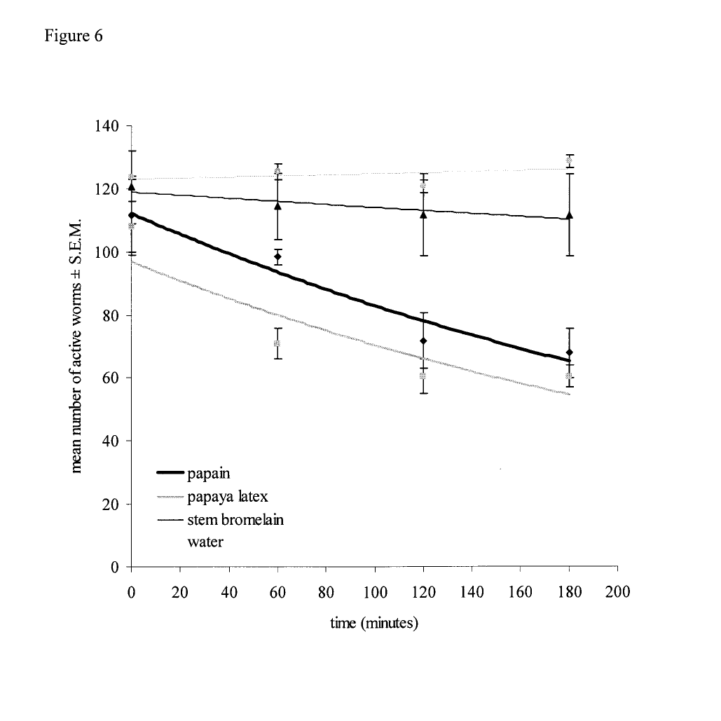

When J2s were incubated without these enzymes, there was no significant reduction in the number of active worms. The effects of papaya latex and papain on the mobility of G. rostochiensis were similar to each other (F_{1,4}=1.96, p=0.234). The effect of stem bromelain, on the other hand, was similar to the controls (F_{1,4}=2.61, p=0.181). When the data for papain and papaya latex were combined, the reduction in mobility was significantly greater overall than the control and stem bromelain combined (F_{1,4}=17.50, p=0.014) (FIG. 6), and there was some evidence that this effect increased with time (F_{1.3,5.1}=6.89, p=0.43). In comparison to Meloidogyne spp., G. rostochiensis J2 needed longer incubation periods with papain and papaya latex before any damage to their cuticle occurred (data not shown).

Effect of Plant Cysteine Proteinases on Meloidogyne spp. Adult Female Worms

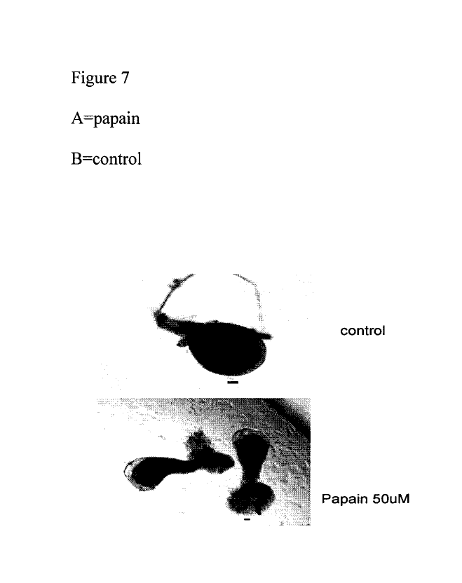

When adult females of M. incognita were incubated with 50 μM (active enzyme) papain, digestion of the cuticle was evident (FIG. 7A). Again, the cuticle of the females incubated with papain differed from that of the worms incubated without enzyme (FIG. 7B) in that the cuticle separated from the nematode after incubation with enzyme, and this cuticle digestion eventually led to the release of the internal structures from these nematodes.

Although the above results demonstrate that plant cysteine proteinases have a nematicidal effect against the J2s and adult worms of sedentary plant parasitic nematodes, these same enzymes had no apparent effect against the eggs of Meloidogyne spp. (results not shown). With light microscopy, examination of the structure of the eggs indicated that they had not been affected. Also, hatching and development of the J2 stage was not affected by incubation of the eggs with plant cysteine proteinases, such as papain.

### Example 2

### Soil Application of Cysteine Proteinases

In the following experiments, we assessed the following parameters:
the nematicidal effect of fruit cysteine proteinases to other sedentary (Heterodera glycines) and migratory (Pratylenchus penetrans) plant parasitic nematodes, and on beneficial nematodes (Steinernema sp and Heterorhabditis sp);
determination of whether the nematicidal effect (concentration dose relationship) is due to nematode paralysis or permanent damage;
determination of whether soil application of these enzymes (concentration dose relationship) to infested soils can affect nematode infection of plants; and
testing whether soil application of these enzymes can render nematodes more vulnerable to harmless concentrations of nematicides.

### Materials and Methods

### Nematodes

Eggs of M. incognita race 1 from North Carolina State University, USA were collected from tomato-infected roots grown in a glasshouse at 23° C. in a 16:8 h light-dark regime. Eggs were extracted using 1% hypochlorite (NaClO) solution (Hussey and Barker, 1973) or by picking egg masses from tomato-infected roots. Juveniles (J2) were hatched from the eggs and egg masses in water. Heterodera glycines eggs were obtained from soil samples stored in Rothamsted-Research. Second stage juveniles (J2) were hatched from the eggs in water after 10 days. Pratylenchus penetrans were a gift from Dr. Deeren Anne-Marie from the Institute for Agricultural and Fisheries Research, Merelbeke, Belgium. The beneficial nematodes Steinernema feltiae and Heterorhabditis megidis were a gift from Dr. Paul Williams from Becker Underwood.

### Cysteine Proteinases

The enzymes used during this study were crude papain from latex of C. papaya (Sigma P3125) (Merops identifier C01.001), stem bromelain from the stem of the pineapple plant, Ananas comosus (Sigma, B4872) (Merops identifier C01.005) and actinidin from New Zealand Pharmaceuticals Limited (Actinidia chinensis; Merops identifier C01.007). These enzyme preparations were standardised for the molar concentration of active cysteine proteinase by titration with the cysteine proteinase-specific inhibitor, L-trans-epoxysuccinyl-leucylamido(4-guanidino)butane (E-64) (Sigma) according to protocol described in Rowan et al., 1988.

### A. In Vitro Assessment of the Activity of Plant Cysteine Proteinases Against Plant Parasitic Nematodes.

Approximately 100 J2s of M. incognita were incubated per well of a 24-well plate with papain, bromelain or actinidin prepared with 16 mM L-cysteine (to activate the cysteine proteinase) dissolved in distilled water. The final volume of the reaction was adjusted to 250 μl with PBS pH 7.2. The active enzyme concentrations used were:

### Papain=250 μM; 125 μM; 62.5 μM; 31.6 μm; 15.5 μM; 7.5 μM

### Bromelain=2.2 mM; 1.1 mM; 560 μM; 280 μM; 140 μM; 70 μM

### Actinidin=3.3 mM; 1.7 mM; 840 μM; 420 μM; 210 μM; 105 μM

Control wells were set up which contained J2s and distilled water with 16 mM L-cysteine. Each enzyme concentration and the controls were set up in duplicate (i.e. n=2). The actual number of active J2s per well was counted with the aid of an Olympus inverted microscope, prior to the addition of the enzymes, and then the J2s were incubated in the dark at room temperature for 2 hs, 16 hs, 24 hs or 40 hr. The number of live and dead J2s per well was counted after each enzyme incubation. The same procedure was followed when setting up further experiments to compare the effects of these enzymes with Heterodera glycines J2, Pratylenchus penetrans, Steinernema feltiae and Heterorhabditis megidis.

### B. Assessment of the Activity of Plant Cysteine Proteinases Against Plant Parasitic Nematodes Present in Soil Mixtures.

The same procedure described above was used to determine the effect of the cysteine proteinases against M. incognita J2 present in soil mixtures. The following soil mixtures were used: fine and coarse sand, peat and fine/coarse sand and loam mix and fine/coarse sand. These soil mixtures were prepared at concentrations of 1:1, 1:3 and 1:6 and autoclaved at 80° C. for 20 min. Peat and loam-mix were obtained from Rothamsted-Research. Soil mixtures were added to 24 well plates (0.5 g) and wetted for 16 hs with either PBS ph 7.2, Tx-100, Milk 5% or cysteine proteinases, in order to wet the soil and block non-specific binding sites present in the soil particles. Then, approximately 100 J2 were added to the wells, followed by cysteine proteinases with 16 mM of L-cysteine to activate the cysteine proteinases or water as control. After 16 hs at room temperature in the dark, individual wells of the 24 well plates were flushed with 1 ml of distilled water to recover the nematodes from the soil mixtures. These were placed in a 24 well plate and the number of dead/live nematodes scored under microscopic observation.

### Nematicides

The following nematicides were used alone or in combination to treat soil infected with M. incognita J2 (see table 2 below):

Oxamyl is supplied as 10% liquid Vydate. 125 μl of a solution 1000 fold dilution of 10% formulation (kindly supplied by DK from NIU) was applied to 5 g of the soil mixture with 16% organic matter, to give a partial nematicidal effect. Soil mixtures were wetted with PBS pH 7.2, 16 hrs prior nematode application of 1000 or 2000 M. incognita J2. Infected soil mixtures were treated with water (control) or 250 μM of active papain in 16 mM of L-cysteine or a combination of Vydate and 250 μM of active papain in 16 mM of L-cysteine. After 48 hs tomato seedlings one month old were added to the treated soils. After 11 days the roots were washed free of soil and chopped to approximately 2 mm pieces. The root pieces of each tomato plant was placed in muslin bags and stained with acid-fuchsin. The stained roots were examined under microscope and all the nematodes present in the roots were counted. In some experiments the soil mixture was wetted with PBS pH 7.2 or 250 μM of active papain for 16 hs to block the non-specific binding site present in the soil particles. After that approximately 1000 M. incognita J2 were applied to soil mixtures, followed by application of 250 μM of active papain in presence of 16 mM of L-cysteine, or water (control) or Vydate alone or a combination of 250 μM of active papain in presence of 16 mM of L-cysteine and Vydate. After 48 hours tomato seedlings one month old were added to the treated soils and after 11 days post infection nematode development inside root tissues was assessed as described above.

The avermectins are macrocyclic lactones produced by the actinomycete Streptomyces avermitilis. Abamectin (Sigma, 46392) has been shown to be an effective nematicide in sandy or low organic soils. According to Cayrol et al., 1993, application of 2000 of abamectin solution (100 mg/l) to 50 g of infected soil allows 75% of larvae to develop and 300 μl allowed 0% larvae development. The same concentrations of abamectin reported in this paper were used to assess the effect of soil treatment with abamectin combined with papain from papaya latex on the penetration of tomato roots by M. incognita. Abamectin was applied to the soil mixtures 2 days prior nematode infection. Using the same protocol described above, 5 g of soil mixture with 1.6% organic matter was firstly wetted for 16 hs with PBS pH 7.2 or 250 μM papain. Approximately 1000 M. incognita J2 were then applied to the soil mixture and then either water (control) or 20 μl or 30 μl of abamectin alone or a combination of 20 μl of abamectin with a mix of 250 μM of active papain and L-cysteine was added. After 48 hs tomato seedlings one month old were added to the treated soils and after 11 days post infection nematode development inside root tissues was assessed as described above.

C. In Planta Assessment of Nematode Invasion of Tomato Plants in Infected Soil Mixtures Treated with Cysteine Proteinases.

### 1) One Soil Application of Cysteine Proteinases

Approximately 2000 J2 of M. incognita were added to 5 g of 1:6 loam and sand which was previously wetted for 16 hs with PBS pH 7. Then a solution of 300 μM or 150 μM of active papain (in 16 mM of L-cysteine) with and without 125 μl Vydate was added to soil and left to react for 48 hs. After this period of time tomato seedlings one month old were added to the treated soil mixtures and placed in a glasshouse at 23° C. in a 16:8 h light-dark regime (Table 1). After 11 days nematode development in the root tissue was assessed.

TABLE 1
One soil Application

| | Active papain | |
|---|---|---|
| Treatment | concentrations | Vydate |
| Control - water | None | None |
| Papain | 300 μM | None |
| Papain + Vydate | 300 μM | 125 μl |
| Papain | 150 μM | None |
| Papain + Vydate | 150 μM | 125 μl |

### 2) Two Soil Application of Cysteine Proteinases

Approximately 1000 J2 of M. incognita were added to 5 g of 1:6 loam and sand which was previously wetted for 16 hs PBS pH 7.2 containing active cysteine proteinases (250 μM for papain, 2.2 μM for bromelain or 3.3 mM for actinidin). The infected soils were then treated for 48 hs with the different solutions listed in Table 2 (controls, cysteine proteinases in 16 mM of cysteine or 250 μM active papain in 16 mM of L-cysteine combined with nematicide solutions). Each solution applied to the soil mixtures were set up as triplicates (n=3). Tomato seedlings one month old were then added to this treated soils and placed in a glasshouse at 23° C. in a 16:8 h light-dark regime. After 11 days the roots were stained as described above and the number of nematode inside the root determined.

### 3) In Vitro Experiments to Determine the Best Concentration of Vydate to Apply to Infected Soils to Give Partial Nematicidal Effect.

Approximately 20 M. incognita J2 were applied to 0.5 g with 16% OM and placed in wells of 24 well plates. Vydate solution with concentrations of 7.5 μl, 15 μl and 30 μl (from a solution 1000 fold of original 10% supplied) were applied to the soil mixture alone or in combination with active papain 50 μM. After 16 hrs of incubation at room temperature in the dark the number of dead/live nematodes was scored by microscope observation. When 15 ul of vydate solution was incubated with nematodes there was 50% of J2 mortality and the same volume of vydate in combination with papain gave approximately 70% of nematode mortality. Indicating an in vitro beneficial, nematotoxic action between papain and vydate (data not shown).

TABLE 2
Two soil applications with enzymes

| | | | Two soil |
|---|---|---|---|
| | Cysteine | | applications |
| Treatments | proteinases | nematicide | of active papain |
| A (control) | none | None | No (water + PBS |
| | | | pH 7.2) |
| B | papain | None | Yes |
| C | bromelain | None | Yes |
| D | actinidin | None | Yes |
| F | papain | Abamectin | Yes |
| I | none | Abamectin 20 μl | No |
| L | none | Abamectin 30 μl | No |
| G | papain | Vydate 125 μl | Yes |
| J | none | Vydate 125 μl | No |
| M | none | Vydate 250 μl | No |

### Results:

(1) Nematicidal Effect of Cysteine Proteinases to Other Nematodes

(2) Concentration Dose Relationship Determines Whether Nematicidal Effect is Due to Nematode Paralysis or Permanent Damage.

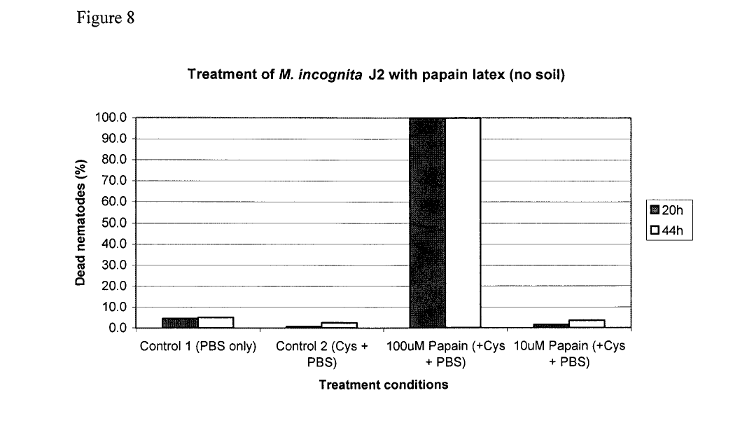

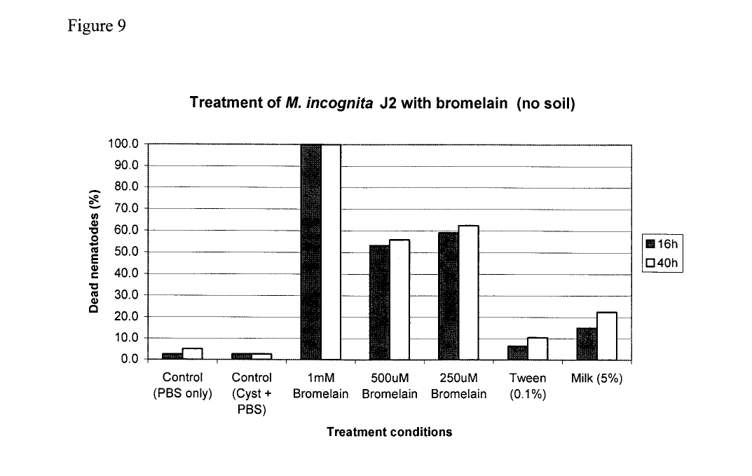

We show that concentrations of 10004 of active papain from papaya latex and 1 mM of active bromelain have a nematicidal effect on M. incognita J2 (in solution without soil) and that the nematodes were not able to recover movement after 40 hs of observation (FIGS. 8 and 9).

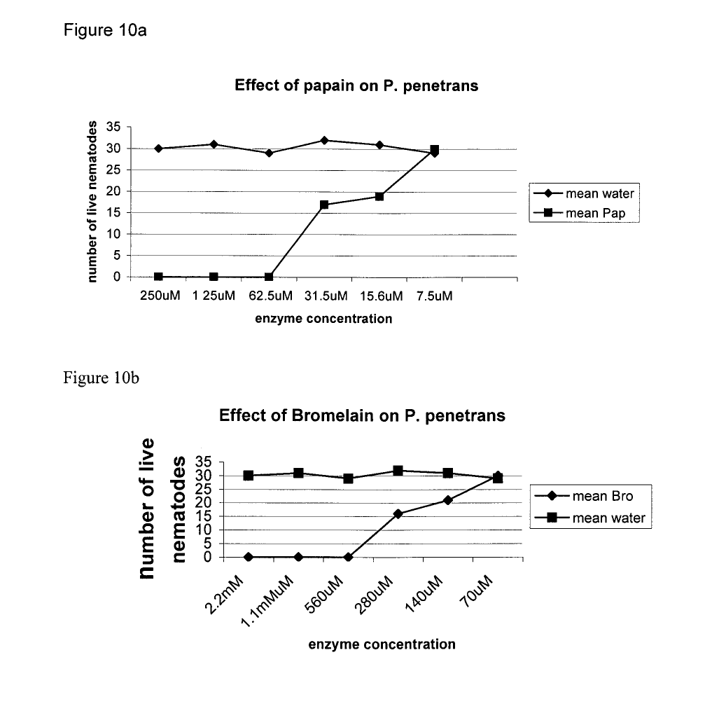

Similar results were obtained for Pratylenchus penetrans and the nematodes were killed by a concentration of 62.504 papain latex and 560 μM bromelain and no recovery of mobility was observed after 48 hs (FIGS. 10a and b)

A small number of Heterodera glycines J2 were treated with 250 μM of active papain from papaya latex and they were dead after 16 hs of incubation and no recovery was observed after 48 hs.

The beneficial nematodes Hetorhabditis megidis and Steinernema feltiae were incubated with dilutions of actinidin, bromelain and pappain from papaya latex (see concentrations used in table 3). These nematodes reacted differentially to these cysteine proteinases (Tables 4 and 5). H. megidis were paralysed in the presence of higher concentrations of actinidin and bromelain but were only affected slightly by the lower concentrations. In contrast, papain did not affect these nematodes to the same extent and at concentrations of 62.4 μM they were moving slowly and were not affected anymore by concentrations of 15 μM. S. feltiae were dead at all concentrations of actinidin tested. In contrast, high concentrations of bromelain affected S. feltiae movement slightly and no effect was observed with concentrations below 280 uM Papain from papaya latex affected S. feltiae movement only at concentrations of 250 uM. After 16 hs of observation the enzyme solutions were diluted with water (to half of the initial concentrations) however, no change in nematode movement was observed after 24 hs.

TABLE 3
ENZYME CONCENTRATIONS

| | ACTINIDIN | BROMELAIN | PAPAIN |
|---|---|---|---|
| A | 3.3 mM | 2.2 mM | 250 uM |
| B | 1.7 mM | 1.1 mM | 125 uM |
| C | 840 uM | 560 uM | 62.5 uM |
| D | 420 uM | 280 uM | 31 uM |
| E | 210 uM | 140 uM | 15 uM |
| F | 105 uM | 70 uM | 7.5 uM |

TABLE 4
Heterorhabditis megidis

| Enzyme | | | Papain from |
|---|---|---|---|
| concentrations | Actinidin | Bromelain | papaya latex |
| A | All paralysed | All paralysed | All moving |
| | | | slowly |
| B | All paralysed | All paralysed | All moving |
| | | | slowly |
| C | All paralysed | All paralysed | All moving |
| | | | slowly |
| D | 50% Moving | 50% moving | No effect |
| | slowly | slowly | nematode motility |
| | | | or behaviour |
| E | All nematodes | All nematodes | No effect |
| | moving slowly | moving slowly | nematode motility |
| | | | or behaviour |
| F | All nematodes | All nematodes | No effect |
| | moving slowly | moving slowly | nematode motility |
| | | | or behaviour |

| | |
|---|---|
| Water | No change in nematode motility or behaviour |

TABLE 5
Steinernema feltiae

| Enzyme | | | |
|---|---|---|---|
| Concentrations | Actinidin | Bromelain | Papain from latex |
| A | All dead | 50% moving | 70% paralysed |
| | | slowly | |
| B | All dead | 50% moving | All moving slowly |
| | | slowly | |
| C | All dead | 50% moving | No effect on |
| | | slowly | nematode motility |
| | | | or behaviour |
| D | All dead | 50% moving | No effect on |
| | | slowly | nematode motility |
| | | | or behaviour |
| E | All dead | No effect on | No effect on |
| | | nematode motility | nematode motility |
| | | or behaviour | or behaviour |
| F | All dead | No effect on | No effect on |
| | | nematode motility | nematode motility |
| | | or behaviour | or behaviour |

| | |
|---|---|
| Water | No change in nematode motility or behaviour |

(3a) Soil Application of Cysteine Proteinases to Infested Soils Reduced Nematode Infection of Plants.

In vitro pilot experiments were designed to determine whether organic matter present in soil would affect the nematicidal properties of the papain from papaya latex. It is often reported that active ingredients of compounds used to control nematodes may become tightly adsorbed by organic matter and become less available for nematode control. Chemical degradation, leaching and biological degradation are also factors that could interfere with the activity of cysteine proteinases in soil. 3a) In vitro assays to determine the effect of papain from papaya latex on M. incognita J2 present in different soil mixtures.

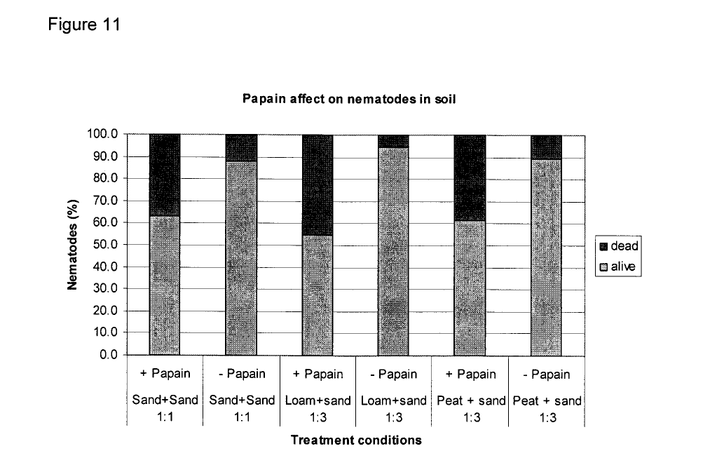

These in vitro experiments were performed in 24 well plates using sterile soil mixtures to diminish the possibility of biological degradation of the enzyme by microorganisms. One hundred freshly hatched J2 of M. incognita were added to 0.5 g of the following soil mixtures: 1:1 coarse sand and fine sand; 1:3 sand and Rothamsted Research loam mix and 1:3 sand and Rothamsted Research peat mixture. These infected soils were then treated with papain latex containing 100 μM of cysteine activity for 16 hours at room temperature in the dark. The number of live nematodes was counted before and after enzyme treatment. The graph shows that all soil mixtures interfered with the nematicidal properties of papain latex causing only 40-50% nematode mortality (FIG. 11).

3b) Attempts to Minimise the Interference of Organic Matter with the Cysteine Activity in Soil

The first approach used was to reduce the amount of organic matter to 16% in the soil mixture (1:6 of loam mix and sand and 1:6 of peat and sand).

The second approach was to identify blocking agents which could block non-specific binding sites in the soil particles. However, the application of detergents such as Tx-100 or Milk (5%) to the soil prior to the application of papain did not deter the loss of enzyme activity when in contact with the soil mixtures (not shown). Triton x-100 (1%) and Milk (5%) slightly affected nematode mobility (FIG. 9).

An alternative approach was to apply papain to the soil twice, the first application of papain (without L-cysteine) would ensure that non-specific binding sites of the soil particles would be blocked. Therefore, the second application of papain with L-cysteine would render the enzyme active. As a further alternative, the concentration of nematoxic agents can be doubled and the soil treated a single time.

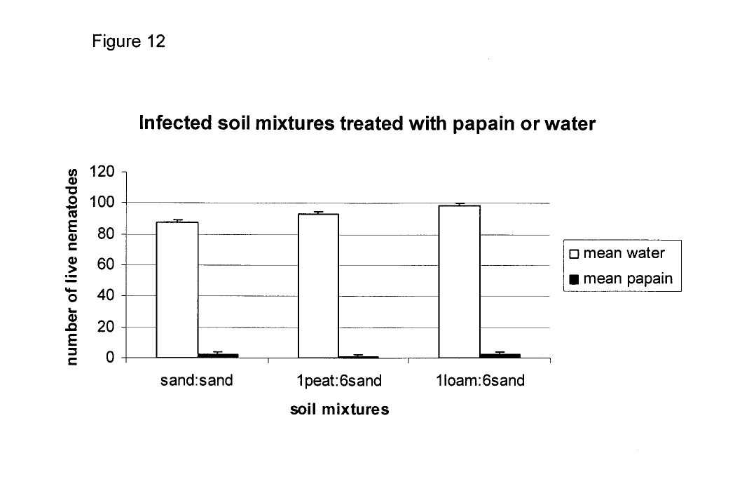

The assay using a soil mixture with 16% of organic matter and applying PBS pH 7.2 with 250 μM of papain (without L-cysteine) to wet the soil worked successfully. After a few hours, approximately 100 M. incognita J2 were added to the soil mixture, followed by a solution of 250 μM of papain in 16 mM of L-cysteine. After 16 hs most nematodes were dead, indicating that the first application of the enzyme must have blocked the soil particles which had previously interfered with the enzyme activity of papain in soil (FIG. 12).

4) In Planta Bioassay Determined that One Application of Cysteine Proteinases to Infested Soils (16% of Organic Matter) Reduced Nematode Infection of Tomato Plants.

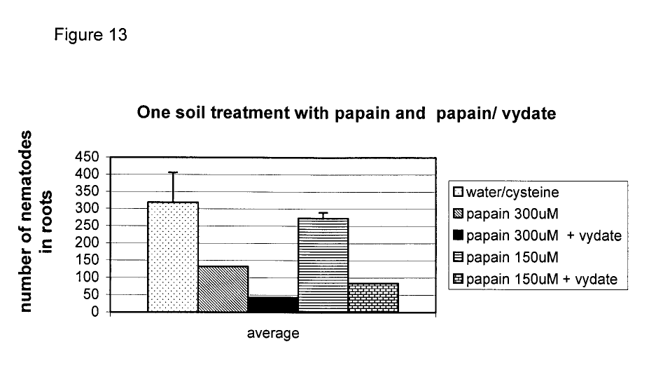

When 300 μM of active papain from papain latex was applied once to the soil mixture with 16% of organic matter (1:6 loam mix and sand), nematode infection of tomato roots was reduced by 60% in comparison with controls. However, when Vydate was added together with 300 μM of active papain the reduction of nematode infection of tomato plants was of 87% in comparison with controls (FIG. 13 and table 6).

When 150 μM of papain was applied once to the soil mixture with 16% of organic matter (1:6 loam mix and sand), nematode infection of tomato roots was reduced by only 14% in comparison with controls. However, when Vydate was added together with 150 μM of papain the reduction of nematode infection of tomato plants was of 74% in comparison with controls (FIG. 13 and table 6).

This shows a beneficial, nematotoxic effect of papain from papaya latex and vydate at concentrations to give partial nematicidal effect.

TABLE 6
Reduction of nematode infection of tomato plants with one
application of papain or papain/vydate to infected soils

| | Mean | |
|---|---|---|
| | number of | Reduction of |
| | nematodes | nematode infection |
| Treatments | in roots | of tomato plants |
| water/L- | 318.5 | 0% |
| cysteine | | |
| papain | 132 | 60% |
| 300 μM | | |
| papain | 43 | 87% |
| 300 μM + | | |
| vydate | | |
| papain | 272 | 14% |
| 150 μM | | |
| papain | 84 | 74% |
| 150 μM + | | |
| vydate | | |

6) In Planta Bioassays Determined that Two Applications of Cysteine Proteinases to Infected Soils (16% of Organic Matter) Reduced Further Nematode Infection of Tomato Plants.

For this experiment 250 μM of papain diluted in PBS pH 7.2 without L-cysteine was firstly applied to soil mixture with 16% of organic matter as a wet and blocking agent.

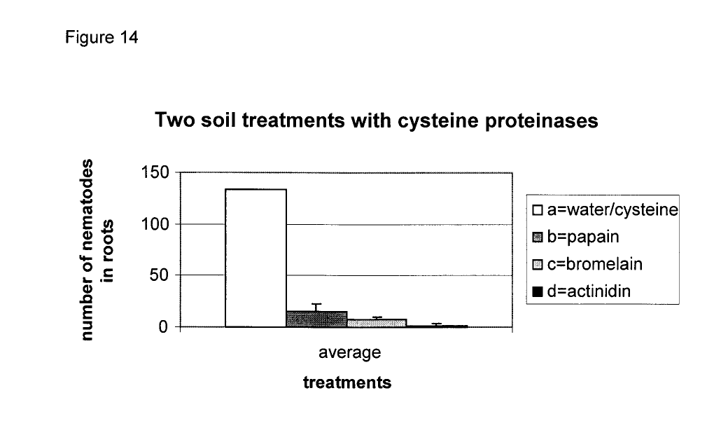

After 16 hrs, 1000 M. incognita J2 were added to the soil, followed by a second application of 250 μM of active papain with L-cysteine. This treatment lasted 48 hrs and then tomato seedlings one month old were added to treated or untreated soil mixtures. Nematode infection of plants was determined 11 days post inoculation. The first application of papain to the soil probably blocked non-specific sites in the soil particles allowing the nematicidal activity of papain to act and reduce nematode infection of plants by 89% for papain, 95% for bromelain and for 99% for actinidin (FIG. 14).

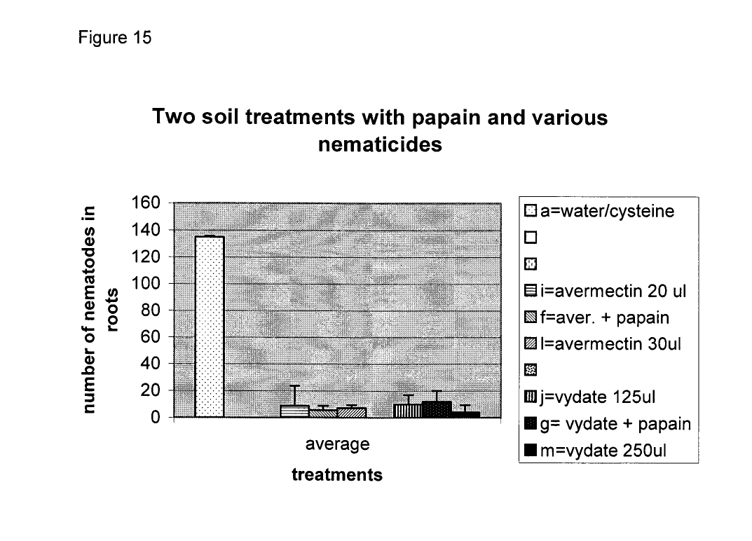

Nematode infection of plants was reduced between 90%-95% when infected soil mixture was treated twice with cysteine proteinases or in combination with nematicides, as shown in FIG. 15.

This experiment shows that cysteine proteinases applied twice in the soil had the same nematicidal effect as Vydate (97%), and abamectin (94%). There were no differences in the reduction of nematode infection between two applications of active papain alone; abamectin and vydate alone or a combination of papain and vydate or papain and abamectin.

### Conclusions:

1) Pratylenchus penetrans and Heterodera glycines were susceptible to the nematicidal effect of papain and bromelain and papain, respectively. Nematode damage was irreversible.

2) One soil (16% organic matter) application of active papain from papaya latex seem to render nematodes more vulnerable to lower concentrations of Vydate with 87% reduction of nematode infection of tomato plants.

3) Two soil (16% organic matter) applications of active cysteine proteinases reduced nematode infection of tomato plants by 90%-95%.

4) Papain affected slightly the movement of the beneficial nematodes H. megidis and S. feltiae.

5) Bromelain also affected slightly the movement of S. feltiae, however H. megidis were paralysed with occasional movement at higher concentrations of bromelain.

6) All concentrations of actinidin were nematicidal towards S. feltiae however, only higher concentrations affected H. megidis.

7) Cysteine proteinases may be used successfully to control plant nematodes when applied to infected soil using a formulation devised to protect/minimise or mask the effect of soil particles on the cysteine proteinases activity.

### REFERENCES

### Mentioned in this Example

Akhtar, M. and Mahmood, I. (1994). Potentiality of phytochemicals in nematode control: a review. Bioresource Technology 48, 189-201.
Berger, J. and Asenjo, C. F. (1939). Anthelmintic activity of fresh pineapple juice. Science 90, 299-300.
Fioretti, L. Porter, A., Haydock, P. & Curtis, R. H. C. (2003). Monoclonal antibodies reactive with secreted-excreted products from the amphids and the cuticle surface of Globodera pallida affect nematode movement and delay invasion of potato plants. International Journal of Parasitology, 32:1709-1718.
Giday, M., Asfaw, Z., Elmqvist, T. and Woldu, Z. (2003). An ethnobotanical study of medicinal plants used by the Zay people in Ethiopia. Journal of Ethnopharmacology 85, 43-52.
Hussey, R. S. and Barker, K. R. (1973). A comparison of methods of collecting inocula of Meloidogyne spp. including a new technique. Plant Disease Report 57, 1025-1028.
Rowan et al. (1988). Arch. Buichem. Biophys., 267: 262-270.
Sharon, E., Spiegel, Y., Solomon, R. & Curtis, R. H. C. (2002). Characterization of Meloidogyne javanica surface coat using antibodies and their effect on nematode behaviour. Parasitology, 125: 177-185.; Stepeck. G., Behnke, J., Buttle, D., Duce, I. R. (2004). Natural plant cysteine proteinases as antihelminthics. Trends in Parasitology, 20, 322-327.

### Example 3

The gene sequences for the various cysteine proteinases referenced in this patent disclosure are known. For example, see UniProtKB/Swiss-Prot entry P00785; Podivinsky E., Forster R. L. S., Gardner R. C.; “Nucleotide sequence of actinidin, a kiwi fruit protease.”; Nucleic Acids Res. 17:8363-8363 (1989); Praekelt U. M., McKee R. A., Smith H.; “Molecular analysis of actinidin, the cysteine proteinase of Actinidia chinesis.”; Plant Mol. Biol. 10:193-202 (1988); Carne A., Moore C. H.; “The amino acid sequence of the tryptic peptides from actinidin, a proteolytic enzyme from the fruit of Actinidia chinensis.”; Biochem. J. 173:73-83 (1978); Keeling J., Maxwell P., Gardner R. C.; “Nucleotide sequence of the promoter region from kiwifruit actinidin genes.”; Plant Mol. Biol. 15:787-788 (1990); the disclosures of each being incorporated by reference herein.

In one embodiment of this invention, transgenic plants are produced with the cysteine proteinases gene from fruits. In one set of plants, expression of the protein is restricted to the apoplast in order to encounter the nematode during penetration or migration inside the roots. The constitutive promoter CaMV35S is suitable for this, although other promoters would also be useful for this purpose. For example, see Lilley, et al. “Preferential expression of a plant cystatin at nematode feeding sites confers resistance to Meloidogyne incognita and Globodera pallida”, Plant Biotechnology Journal, Volume 2 Issue 1 Page 3—January 2004, 1467-7652, herein incorporated by reference, which discloses the expression patterns of three promoters preferentially active in the roots of Arabidopsis thaliana which were investigated in transgenic potato plants in response to plant parasitic nematode infection. Promoter regions from the three genes, TUB-1, ARSK1 and RPL16A were linked to the GUS reporter gene and histochemical staining was used to localize expression in potato roots in response to infection with both the potato cyst nematode, Globodera pallida and the root-knot nematode, Meloidogyne incognita. All three promoters directed GUS expression chiefly in root tissue and were strongly up-regulated in the galls induced by feeding M. incognita. Less activity was associated with the syncytial feeding cells of the cyst nematode, although the ARSK1 promoter was highly active in the syncytia of G. pallida infecting soil grown plants. Transgenic potato lines that expressed the cystatin OcIΔD86 under the control of the three promoters were evaluated for resistance against Globodera sp. in a field trial and against M. incognita in containment. Resistance to Globodera of 70±4% was achieved with the best line using the ARSK1 promoter with no associated yield penalty. The highest level of partial resistance achieved against M. incognita was 67±9% using the TUB-1 promoter. In both cases this was comparable to the level of resistance achieved using the constitutive cauliflower mosaic virus 35S (CaMV35S) promoter. The results establish the potential for limiting transgene expression in crop plants whilst maintaining efficacy of the nematode defence. Interestingly, however, the approach disclosed in the present patent disclosure is essentially the direct opposite of what is disclosed in this cited reference, as here we disclose nematode control by expression of cysteine proteinases, whereas Lilley, et al. disclose nematode control via expression of cystatins, which are cysteine proteinase inhibitors. Nonetheless, in an analogous fashion, the enzymes according to the present invention may be expressed in plants for the purposes of conferring nematode resistance as disclosed herein.

Likewise, reference may be made to U.S. Pat. No. 5,977,440, which discloses a method of conferring insect resistance on a plant by expression of a maize cysteine proteinase in the plant. Likewise for U.S. Pat. No. 6,228,643, herein incorporated by reference, which discloses an oilseed rape cysteine proteinase promoter and methods of use thereof to control expression of genes in plants. Likewise for U.S. Pat. No. 7,049,484 which discloses methods and compositions for commercial production of proteases in plants without damage to the plants. In order to practice the present invention, those skilled in the art may follow the teachings and disclosure in the '484 patent for purposes of growing nematode resistant plants, rather than for purposes of commercial production of proteases.

### Example 4

Barley cysteine endoproteinases (EP) EP-A and EP-B are secreted by the scutellum and aleurone layer into the starchy endosperm during germination, they play a central role in the breakdown of barley (Hordeum vulgare L.) endosperm storage proteins (hordeins).

EP-B2 is secreted as a proenzyme (proEP-B2) into the acidic endosperm of germinating barley seeds, where it is activated to its natural form. Its physiological substrates are Pro- and Gln-rich hordeins, which are major storage proteins in barley gluten (Bethune et al., 2006). Substrate specificity analysis of EP-B2 suggests it cleaves preferentially after Gln residues, often with a Pro at the position P2 (Davy et al., 1998).

A sample of the recombinant barley proteinase EP-B2 was obtained from Prof. Chaitan Khosla from Stanford University, which was produced in their lab by introducing the plasmid PMtb1, which encodes proEP-B2, in E. coli (Bethune et al, 2006).

The effect of this recombinant proteinase was tested on J2 of Meloidogyne incognita and the enzyme proved to be nematotoxic to this nematode. A double dilution starting with dilution 1:1 of the recombinant EP-B2 in assay buffer at pH 4 (citrate-phosphate buffer pH 4, containing 2 mM of L-cysteine) was utilized.

Controls were:

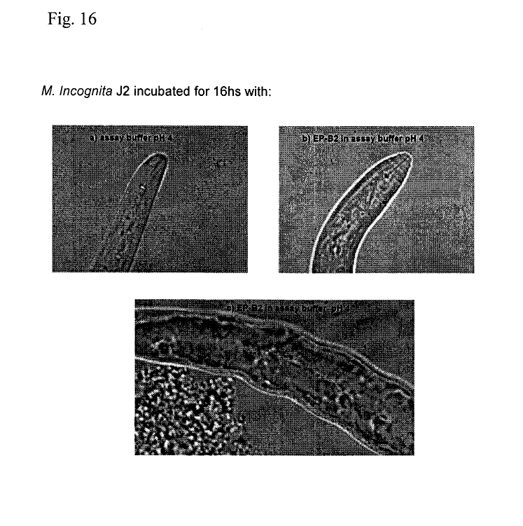

1) Nematodes incubated in assay buffer at pH 4 with no recombinant enzyme. No effect was observed in the nematodes (FIG. 16a).

2) Nematodes incubated with EP-B2 only (without the assay pH 4 buffer), to show whether the recombinant protein had any nematotoxic effect unrelated to the enzymatic activity. No effects on the nematodes were observed.

3) Nematodes incubated with another assay buffer with a neutral pH (PBS pH 7 with 2 mM of L-cysteine). A marginal effect was noted on the nematodes, as EP-B2 activity is much reduced at neutral pH.

The results show that the recombinant EP-B2 diluted 1:1, in the pH 4 assay buffer (FIGS. 16a and 16b) but not pH 7, damages the cuticle of J2 of M. incognita and that the nematodes appeared dead after 2 hrs of incubation. There was no recovery of nematode movement even after 48 hrs.

It is important to note that soil application of cysteine proteinase requires concentrations of cysteine proteinases that are nematotoxic. We have shown in bioassays that soil application of cysteine proteinases at concentrations that kill nematodes prevent nematode infection of plants.

In contrast, lower concentrations of cysteine proteinases, secreted from transgenic plants into the rhizosphere or root tissue, should be effective to cause nematode immobility which would be sufficient to affect nematode invasion of plants. This observation is supported by previous work. See Fioretti et al., 2002; Sharon et al., 2002; GB patent filling 46082 which showed that affecting nematode mobility can interfere with nematode infection of plants.

As the nematode cuticle represents a key target for nematode control, plants over-expressing cysteine proteinases in the rhizosphere should demonstrate increased resistance to nematode infection based on the studies described above. Furthermore, the expression of cysteine proteinases in a cell specific manner (lateral root cap/epidermal cells) allows an elegant method for the control of the nematodes at the site of infection with minimal modification of the plant/crop.

Thus, transgenic Arabidopsis plants ubiquitously expressing the cysteine proteinases described herein will be created to paralyse nematodes or affect their behaviour to reduce nematode invasion of plant roots and their migration within roots. We will then proceed to the next stage, expression in specific cell types within the root.

To produce transgenic Arabidopsis expressing barley EP-B2 cysteine proteinases, binary constructs that place the EP-B2 cDNA under the control of the constitutive 35S promoter will initially be synthesized. Arabidopsis plants will then be transformed with these vectors. The nematicidal effects of the transgenic Arabidopsis plants will then be assessed. While the 35S promoter will be employed initially, a variety of different plant promoters which direct expression in different locations and cell types in the roots will also be utilized.

Expression of the cysteine proteases of the invention can be restricted to the apoplast, thereby encountering and inhibiting nematode infestation during penetration or migration inside roots. The proteases may be expressed and secreted from the epidermal cell to control nematodes at the site of infection. Expression in the lateral root cap or root border cells is desirable to deliver secreted cysteine proteinases into the rhizosphere and stop or inhibit nematode infection of roots. In certain instances, the cysteine proteinases can be expressed near the vascular cylinder (for example in the pericycle and endodermis) where nematodes preferentially form feeding cells. A similar approach has been described by Liu et al. who expressed synthetic chemodisruptive peptides in planta. See Liu et al. 2005.

In another approach, the cysteine proteases may be expressed and secreted from crop seeds into the rhizosphere to reduce crop damage.

Arabidopsis root specific promoters include, without limitation, CAPRICE (EMBL Accession no. AC006526), Root cap 1 (RCP1; EMBL Accession no. AF168391), also the promoters of the enhancer-trapped genes in the following GAL4 enhancer trap lines, e.g., Endodermis/cortex—Enhancer trap line J0571, Endodermis/root cap—Enhancer trap line J2672, and Epidermis+root cap—Enhancer trap line J0481 are suitable. For information on these promoters, see the world wide web at plantsci.cam.ac.uk/Haseloff/construction/catalogFrame.html.

Further experiments include using extracts from the transgenic plants so produced on plant parasitic nematodes Meloidogyne sp. in order to assess their effectiveness as nematotoxic agents.

Clearly, the cysteine protease sensitive cuticle and/or amphidial proteins and the nucleic acids encoding them provide ideal targets for the development of transgenic plants which also exhibit resistance to nematode infection. Disruption of chemoreception and modification of nematode cuticle alter nematode behaviour and suppress its ability to invade roots successfully. See GB Patent Application 46082, Fioretti et al., 2002; and Sharon et al., 2002.

Thus, any of the aforementioned molecules provide suitable targets for the development of silencing RNA molecules including siRNA, shRNA and the like which can be expressed in the root tissue and rhizosphere as described above. The cuticle and amphids are easily targeted as they are exposed to the environment.

### Example 5

Nematode infestation of turf grasses results in costly and unsightly damage, particularly when present on golf course greens. Thus, another aspect of the invention entails the treatment of turf grasses, particularly those used frequently on golf courses, and particularly the golf-course green, using the cysteine proteinases described herein. A drench application protocol for this purpose is provided below.

Soil containing nematodes (e.g., lance, sting nematodes) are obtained from greenhouse cultures. 5 replicate samples are treated with four solutions, enzyme solutions (C1 to C3) at three increasing concentrations plus control (C4)

Typically, 135 cm3 of soil is added to each of 20 (5 replicates each for the four treatments) bags/pots. Solutions a), b) and c) are added once for 2 consecutive days consecutives (days 1 and day 2):
a) Add 5 ml of solution A as wetting agent
b) Add 4 ml solution B per replicate
c) Add 20 ml solution C(C1, C2, C3, C4 as appropriate)
   i) For treatment 1: C1
   ii) For treatment 2: C2
   iii) For treatment 3: C3
   iv) For controls: C4

### Solutions:

Solution A=PBS 0.1M pH.7=wetting solution
Solution B=16 mM Enzyme cofactor (100 ml PBS+1.94 g Enzyme cofactor)
Solution C=Enzyme (total volume needed for each solution for 5 replicates×1 treatments with 2 applications=200 ml)
C1=Enzyme 1x (14 g of Enzyme in 250 ml of water)
C2=Enzyme 2x (28 g of Enzyme in 250 ml of water)
C3=Enzyme 3x (56 g of Enzyme in 250 ml of water)
C4=PBS 0.1M pH 7

On day 3 nematodes are extracted and viability assessed.

Field Application: Cysteine proteinases are applied to existing turf grasses wherein reduction or control of nematode populations is desirable. In this aspect, periodic drenches of, particularly of the golf-course green, is achieved by mixing appropriate concentrations of cysteine, cysteine proteinase and water, with periodic watering and fertilization of the golf-course green. To assist in penetration, the golf-course green may be aerated before, during or after application of such drenches to permit optimal penetration of enzyme and cofactor (cysteine) to the site of nematode attack at the grass roots. The application of enzyme and cofactor is included in irrigation of the golf-course green in a particular geographic location based on the degree of susceptibility to nematode attack of the green. Thus, a single application per season may be sufficient in areas of low susceptibility, while in areas exhibiting a high incidence of nematode damage, multiple drenches per season, or per day, may be used to minimize or eliminate nematode damage. Appropriate concentrations of enzyme and cofactor may be determined by routine experimentation in a particular venue to achieve maximal cost-benefit ratios without undue experimentation. Grasses so treated are resistant to nematode infestation when compared to untreated grasses.

Alternatively, turf grasses are genetically engineered to express the cysteine proteinases described herein prior to planting or seeding. The skilled person is aware of many methods for introducing heterologous nucleic acids into plant cells on interest. See for example U.S. Pat. No. 6,709,867, WO2007/087279 and Molecular Breeding of Forage and Turf Proceedings of the 3rd International Symposium, Molecular Breeding of Forage and Turf, Dallas, Tex., and Ardmore, Okla., U.S.A., May, 18-22, 2003.

Genetically engineered turf grasses which express the cysteine proteinases described herein exhibit enhanced resistance to nematode infestation and prevent the attainment of threshold levels of infestation.

### REFERENCES

Ahmad, M. S., Mukhtar, T. and Ahmad, R. (2004). Some studies on the control of citrus nematode (Tylenchus semipenetrans) by leaf extracts of three plants and their effects on plant growth variables. Asian Journal of Plant Sciences 3, 544-548.
Akhtar, M. and Mahmood, I. (1994). Potentiality of phytochemicals in nematode control: a review. Bioresource Technology 48, 189-201.
Berger, J. and Asenjo, C. F. (1939). Anthelmintic activity of fresh pineapple juice. Science 90, 299-300.
Berger, J. and Asenjo, C. F. (1940). Anthelmintic activity of crystalline papain. Science 91, 387-388.
Chitwood, D. J. (2003). Research on plant-parasitic nematode biology conducted by the United States Department of Agriculture—Agricultural Research Service. Pest Management Science 59, 748-753.
Dubois, T., Jacquet, A., Schnek, A. G. and Looze, Y. (1988). The thiol proteinases from the latex of Carica papaya L. I. Fractionation, purification and preliminary characterisation. Biological Chemistry Hoppe-Seyler 369, 733-740.
Fioretti, L., Porter, A., Haydock, P. and Curtis, R. H. C. (2003). Monoclonal antibodies reactive with secreted-excreted products from the amphids and the cuticle surface of Globodera pallida affect nematode movement and delay invasion of potato plants. International Journal of Parasitology 32, 1709-1718.
Giday, M., Asfaw, Z., Elmqvist, T. and Woldu, Z. (2003). An ethnobotanical study of medicinal plants used by the Zay people in Ethiopia. Journal of Ethnopharmacology 85, 43-52.
Hussey, R. S. and Barker, K. R. (1973). A comparison of methods of collecting inocula of Meloidogyne spp. including a new technique. Plant Disease Report 57, 1025-1028.
Kowlessur, D., O'Driscol, M., Topham, M., Templeton, W., Thomas, E. W. and Brocklehurst, K. (1989). The interplay of electrostatic fields and binding interactions determining catalytic-site reactivity in actinidin. Biochemical Journal 259, 443-452.
McDowall, M. A. (1970). Anionic proteinase from Actinidia chinensis: preparation and properties of the crystalline enzyme. European Journal of Biochemistry 14, 214-221
Meyer, S. L. F. (2003). United States Department of Agriculture—Agricultural Research Service research programs on microbes for management of plant-parasitic nematodes. Pest Management Science 59, 665-670.
Miller, P. M. and Sands, D. C. (1977). Effects of hydrolytic enzymes on plant-parasitic nematodes. Journal of Nematology 9, 192-197.
Robbins, B. H. (1930). A proteolytic enzyme in ficin, the anthelmintic principle of Leche de Higueron. Journal of Biological Chemistry 87, 251-257.
Rowan, A. D., Buttle, D. J. and Barrett, A. J. (1990). The cysteine proteinases of the pineapple plant. Biochemical Journal 266, 869-875.
Sgarbieri, V. C., Gupte, S. M., Kramer, D. E. and Whitaker, J. R. (1964). Ficus enzymes. I: Separation of the proteolytic enzymes of Ficus carica and Ficus glabrata latices. Journal of Biological Chemistry 239, 2170-2177.
Sharon, E., Spiegel, Y., Solomon, R. and Curtis, R. H. C. (2002). Characterisation of Meloidogyne javanica surface coat using antibodies and their effect on nematode behaviour. Parasitology 125, 177-185.
Siddiqi, M. A., Haseeb, A. and Alam, M. (1992). Control of plant-parasitic nematodes by soil amendments with latex bearing plants. Indian Journal of Nematology 22, 25-28.
Stepek, G., Buttle, D. J., Duce, I. R., Lowe, A. and Behnke, J. M. (2005). Assessment of the anthelmintic effect of natural plant cysteine proteinases against the gastrointestinal nematode, Heligmosomoides polygyrus, in vitro. Parasitology 130, 203-211.
Stepek, G., Lowe, A. E., Buttle, D. J., Duce, I. R. and Behnke, J. M. (2006). In vitro and in vivo anthelmintic efficacy of plant cysteine proteinases against the rodent gastrointestinal nematode, Trichuris muris. Parasitology 132, 681-689.
Stepek, G. Curtis R H C, Kerry B R, Shewry P R, Clark S J Lowe A E, Duce I R, Buttle D J, Behnke J M. (2007a). Nematicidal effects of cysteine proteinases against sedentary plant parasitic nematodes Parasitology, 134: 1831-1838.
Stepek, G., Lowe, A. E., Buttle, D. J., Duce, I. R. and Behnke, J. M. (2007b). Anthehelmitic action of plant cysteine proteinases against the rodent stomach nematode, Protospirura muricola, in vitro and in vivo. Parasitology, 134: 103-112.
Sundararaju, P., Guljar, B. and Ratnakaran, K. (2003). Abstracts of the 4^{th} International Workshop in Biological Control in the Tropics, pp 1-3.
Bethune et al., 2006. Heterologous expression, purification refolding and structural-functional characterization of EP-b2, a self-activating barley cysteine endoproteinase. Chemistry and Biology, 13: 637-647.
Davy et al., 1998. Substrate specificity of barley cysteine endoproteinase EP-A and EP-B. Plant Physiology, 117: 255-261.
Fioretti L., Porter A., Haydock P. J. & Curtis R. H. C. Monoclonal antibodies reactive with secreted-excreted products from the amphids and the cuticle surface of Globodera pallida affect nematode movement and delay invasion of potato roots. International Journal of Parasitology, 2002, 32:1709-1718.
Sharon E., Spiegel Y., Solomon R. & Curtis R. H. C. Characterisation of Meloidogyne javanica surface coat using antibodies and their effect on nematode behaviour. Parasitology, 2002, 125: 177-185.
Liu et al. The production of synthetic chemodisruptive peptides in planta disrupts the establishment of cyst nematodes. Plant Biotechnology Journal, 2005 3: 487-496.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of controlling nematode infestation of plants which comprises contacting nematodes with a plant cysteine proteinase or fragments thereof, plus an effective amount of a reducing agent to ensure activity of said cysteine proteinase.

2. The method according to claim 1 wherein said nematode is a plant or animal nematode.

3. The method according to claim 2 wherein said nematode is a sedentary plant parasitic nematode.

4. The method according to claim 3 wherein said cysteine proteinase is derived from a plant.

5. The method according to claim 4 wherein said cysteine proteinase is derived from the latex or fruit of a plant.

6. The method according to claim 5 wherein said plant is selected from the group consisting of papaya, kiwi, fig, barley and pineapple.

7. The method according to claim 6 wherein said plant is kiwi.

8. The method according to claim 6, wherein said plant is barley.

9. The method according to claim 3 wherein said nematodes are sedentary plant parasitic nematodes of the genera Meloidogyne and Globodera.

10. The method according to any one of the previous claims in which said cysteine proteinase is contacted with said nematodes in the presence of a non-enzymatic nematicide.

11. The method according to claim 1 wherein a nucleic acid encoding said cysteine proteinase is introduced and expressed in a plant producing a transgenic plant, wherein said trangenic plant produces cysteine proteinase in sufficient quantity to render the plant more resistant to parasitism by nematodes when compared to a non-transgenic plant.

12. The method according to claim 11, wherein said nucleic acid is isolated from kiwi or barley.

13. The method of claim 1, wherein said plant is turf grass and said nematode is a lance or a sting nematode.

14. A nematotoxic composition comprising at least one plant cysteine proteinase or active fragments thereof, a carrier and an effective amount of a reducing agent.

15. The composition of claim 14 further comprising growing media or soil, said composition being nematotoxic to sedentary plant parasitic nematodes.

16. A vector, plasmid, phage or construct comprising a nucleic acid encoding at least one plant cysteine proteinases or functional fragments thereof, wherein expression of said proteinase in said plant is effective to inhibit growth of plant parasitic nematodes.

17. The vector, plasmid, phage or construct of claim 16, wherein said nucleic acid is isolated from kiwi or barley.

18. The method of claim 1, wherein said contact is effective to confer resistance to plant parasitic nematode before or during or upon initiation of parasitism of said plant by said nematodes.

19. The method according to claim 18 wherein said cysteine proteinase is kiwi cysteine proteinase or barley EP-B2 cysteine protease.

20. A plant transformed with a vector, plasmid, phage or construct of claim 16 wherein said proteinase or functional fragment thereof is operably linked to a tissue specific promoter.

21. The plant according to claim 20 wherein said tissue specific promoter drives expression of said proteinase in the roots of said plant.

22. The plant according to claim 21 wherein said promoter is selected from the group consisting of CAPRICE, Root cap 1 (RCP1), Endodermis/cortex—Enhancer trap line J0571, Endodermis/root cap—Enhancer trap line J2672, and Epidermis+root cap—Enhancer trap line J0481.

23. The method of claim 1, further comprising contacting said nematodes with an effective amount of a nematicide.

24. The method according to claim 23 wherein said cysteine proteinase is selected from the group consisting of papain, actinidin, bromelain and combinations thereof.

25. The method according to claim 23 wherein said nematicide is Vydate.

26. The method according to claim 23 which comprises two administrations of cysteine proteinase containing material, with or without a non-enzymatic nematicide.

27. The method of claim 1, wherein said nematodes are contacted in soil wherein said soil is concurrently treated with or is pre-treated with a composition which protects, minimises or masks the inhibitory effect of soil particles on the cysteine proteinases activity when compared to the absence of concurrent or pre-treatment of said soil with said composition.

28. The plant of claim 20, wherein said proteinase is EP-B2 from barley.

29. The method of claims 23-27 wherein said nematode is a lance or sting nematode and said plant is a turf grass.

30. The method according to claim 1, wherein said plants are contacted with said cysteine protease at sub lethal levels, wherein said levels are effective to inhibit mobility or infective capacity of said nematode.

31. The method according to claims 1 and 14 wherein said reducing agent is cysteine.
